(19) United States
(12) Patent Application Publication
(10) Pub. No.: US 2026/0265221 A1
(43) Pub. Date: Sep. 10, 2026

(54) PARP INHIBITOR, AND PREPARATION METHOD THEREFOR AND MEDICAL USE THEREOF

(71) Applicant: WIGEN BIOMEDICINE TECHNOLOGY (SHANGHAI) CO., LTD., Shanghai (CN)

(72) Inventors: Yuli XIE, Shanghai (CN); Yongcong LV, Shanghai (CN); Lihui QIAN, Shanghai (CN)

(21) Appl. No.: 19/166,407

(22) PCT Filed: Apr. 12, 2024
(86) PCT No.: PCT/CN2024/087460

§ 371 Date: Sep. 17, 2025

(30) Foreign Application Priority Data
Apr. 14, 2023 (CN) 202310401050.X
Jul. 12, 2023 (CN) 202310853866.6

(51) Int. Cl. C07D 401/12; A61K 31/498; A61P 35/00

(52) U.S. Cl. CPC C07D 401/12; A61K 31/498; A61P 35/00

(57) ABSTRACT

Disclosed in the present invention are a PARP inhibitor, a preparation method therefor and pharmaceutical use thereof. Particularly, the present invention relates to novel compounds of general formulas (1) and/or a pharmaceutically acceptable salt thereof, a composition comprising the compounds of general formulas (1) and/or the pharmaceutically acceptable salt thereof, a preparation method therefor and use thereof as a PARP inhibitor in the preparation of anti-tumor drugs.

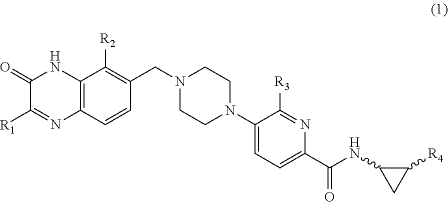

The present application claims priority to Chinese Patent Application No. 202310401050.X filed on Apr. 14, 2023 and Chinese Patent Application No. 202310853866.6 filed on Jul. 12, 2023, all of which are incorporated herein by reference in its entirety.

### TECHNICAL FIELD

The present invention relates to the field of pharmaceutical chemistry, and particularly to a novel compound having an inhibitory effect on poly (ADP-ribose) polymerase (PARP), a preparation method therefor and use of such compounds in preparing anti-tumor drugs.

### BACKGROUND

Poly (ADP-ribose) polymerase (PARP) is expressed in most eukaryotic cells, and takes nicotinamide adenine dinucleotide (NAD+) as a substrate to catalyze the formation of ADP-ribose units or polymers thereof (PARs) on itself or other specific amino acid residues of proteins, so as to modify the PARs of the proteins and further regulate the degradation and functions of the proteins. The PARP family consists of 7 isoenzymes, including PARP-1, PARP-2, PARP-3, PARP-4 (Vault-PARP), tankyrases such as PARP-5 (TANK-1, TANK-2 and TANK-3), PARP-7 and PARP-10 [de la Lastra C A. et al., Curr Pharm Des., 13(9), 933-962, 2007]. Although the PARP family is diverse in enzymes, PARP-1 is responsible for more than 90% of ADP-ribosylation (PAR) within cells. PARPs play important roles in events including chromosome structure regulation, gene transcription, DNA replication and recombination, and DNA repair. Among them, PARP-1 promotes ADP-ribosylation and polymerization, initiates DNA repair and regulates the recruitment and level of DNA repair proteins during DNA repair.

When DNA of tumor cells is damaged by chemotherapy drugs or ionizing radiation, etc., PARP-1 is quickly activated, the NAD+ is taken as the substrate, and a large amount of PARs are synthesized at a DNA damage part to modify histones. DNA repair proteins are then recruited to initiate DNA repair. PARP-1 is mainly involved in the repair of single strand DNA breaks (SSBs). When PARP-1 is inhibited by a PARP inhibitor, SSBs cannot be repaired, and during S-phase DNA replication, the SSBs are converted into double strand breaks (DSBs), thereby inhibiting the function of PARP-1 and leading to accumulation of DSBs in the cells. Repair of the DSBs by the body is mainly achieved in two manners: homologous recombination (HR) and non-homologous DNA end joining (NHEJ), wherein repair by homologous recombination is a main manner of S-phase DSB repair, and the repair reliability is high. BRCA1 and BRCA2 play an important role in repair by the homologous recombination. The BRCA1 and BRAC2 deficiency results in limited DSB repair. The research shows that BRCA1/2 mutation is found in ovarian cancer, breast cancer and prostate cancer, and the cancer cells with BRCA1 and BRCA2 deficiency are particularly sensitive to the PARP inhibitor. This may result from inhibition of PARP-1 by the PAPR inhibitor, the DSBs are induced, and due to BRCA1/2 deficiency, repair is inhibited, thereby inducing cell death. Therefore, the PARP inhibitor has a good effect on clinical treatment of tumors with BRCA1/2 deficiency. The PARP inhibitor can be used alone or in combination with chemotherapy drugs and radiotherapy drugs, thereby achieving the purposes of reducing dosage and improving efficacy. Olaparib is the first PARP inhibitor approved for marketing. With the expanding indications of the PARP inhibitor, the PARP inhibitor is also used extensively, not only around tumors, but also has certain effects on stroke, myocardial ischemia, inflammation and diabetes.

Although efforts to develop the PARP inhibitor for the treatment of cancer and other diseases are ongoing, such inhibitors lack certain specificity for PAPR-family proteins, have non-negligible toxic and side effects, and are prone to drug resistance. Therefore, the development of specific and highly active PARP-1 inhibitors is of important clinical value.

### SUMMARY

The present invention provides a compound of general formula (1), or an isomer, a crystalline form, a pharmaceutically acceptable salt, a hydrate, or a solvate thereof:

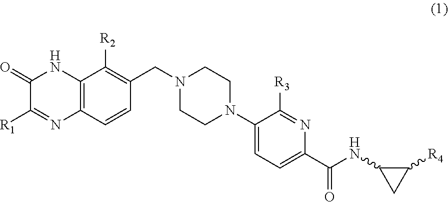

wherein in general formula (1):
R₁ is (C₁-C₃) alkyl, (C₁-C₃) fluoroalkyl, or (C₃-C₄) cycloalkyl;
R₂ is H or F;
R₃ is H, D, halogen, (C₁-C₃) alkyl, or (C₁-C₃) fluoroalkyl;
R₄ is H or F;
when R₄ is F, fluorocyclopropyl of

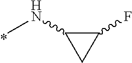

is a single isomer containing two chiral centers.

In another preferred embodiment, in general formula (1),

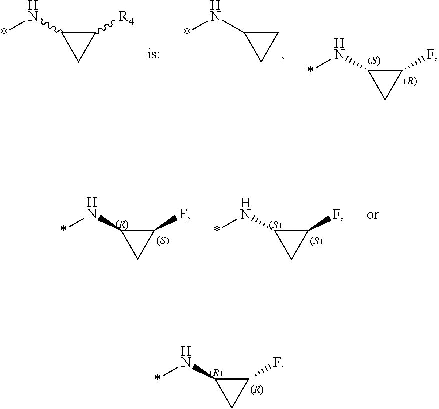

In another preferred embodiment, in general formula (1), R₁ is methyl, ethyl, or cyclopropyl, preferably methyl or ethyl, and more preferably methyl.

In another preferred embodiment, in general formula (1), R₃ is H, D, F, Cl, or methyl, preferably H, D, F, or methyl, more preferably H, D, or F, and more preferably F.

In another specific embodiment of the present invention, the compound of general formula (1) has one of the following structures:

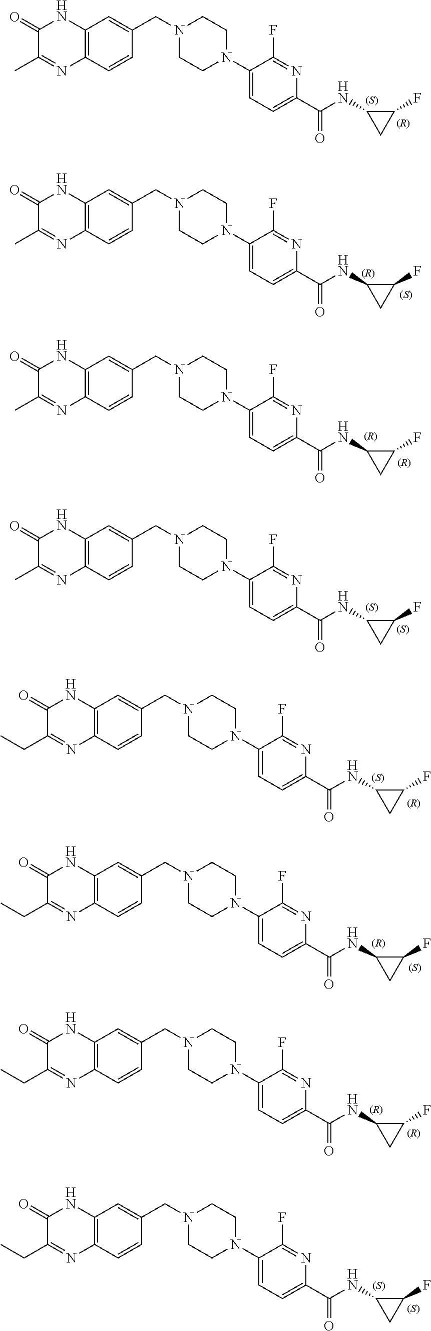

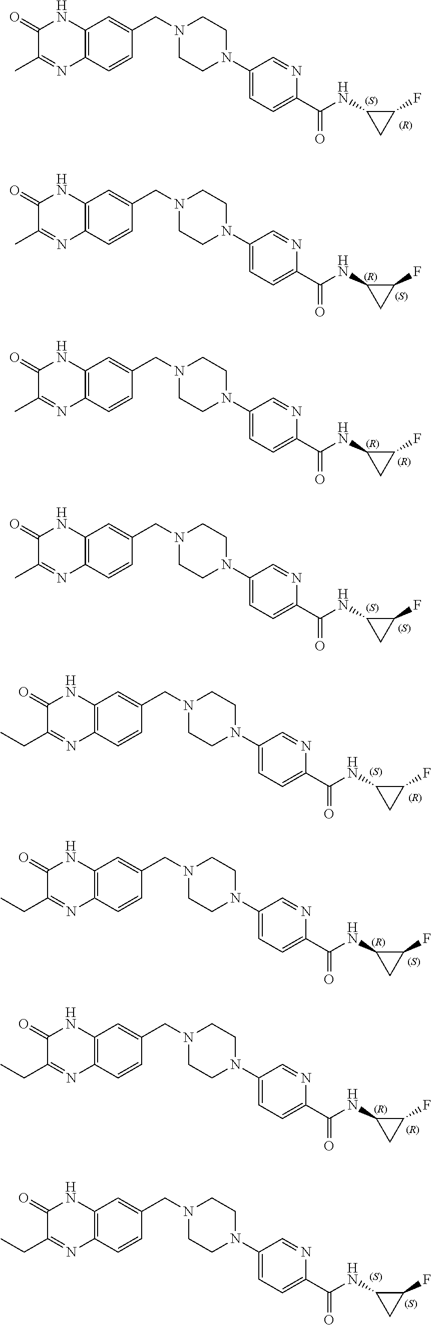

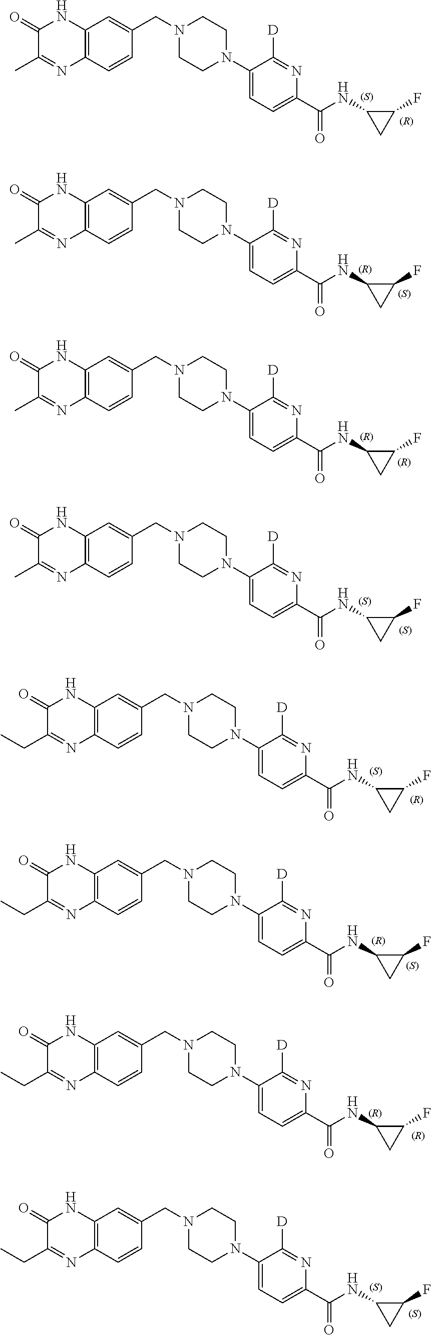

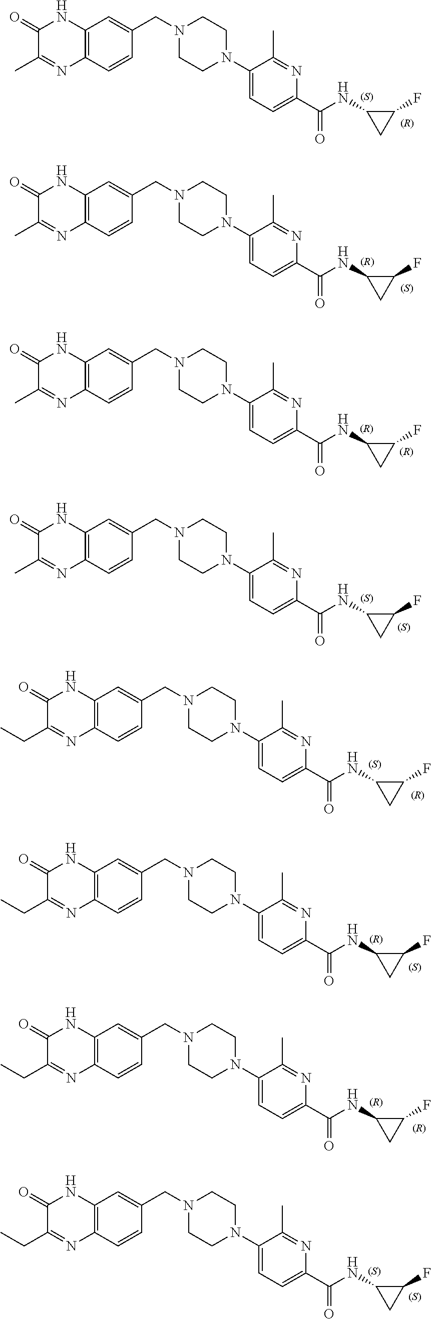

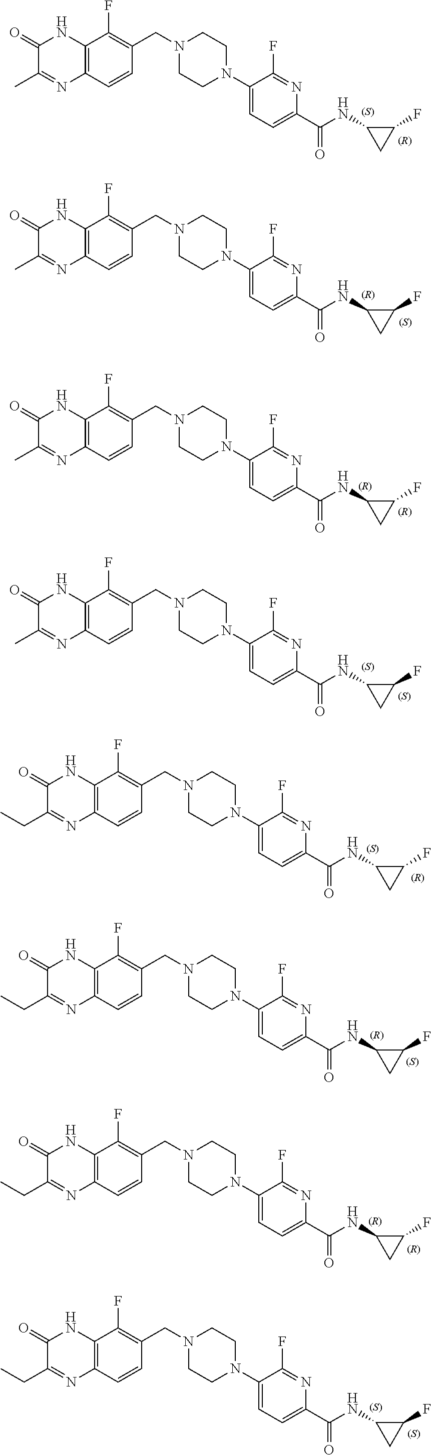

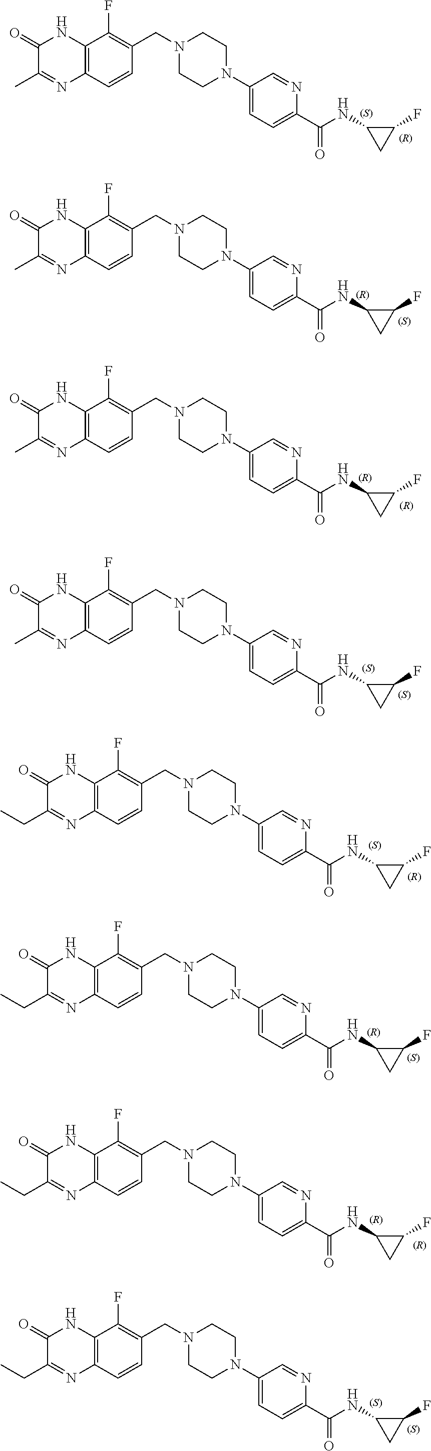

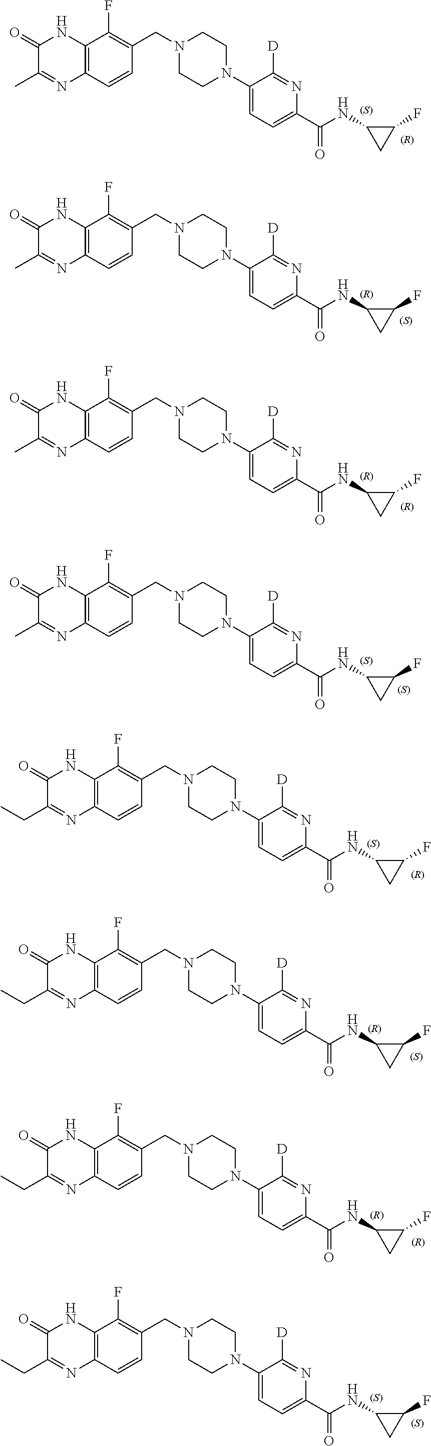

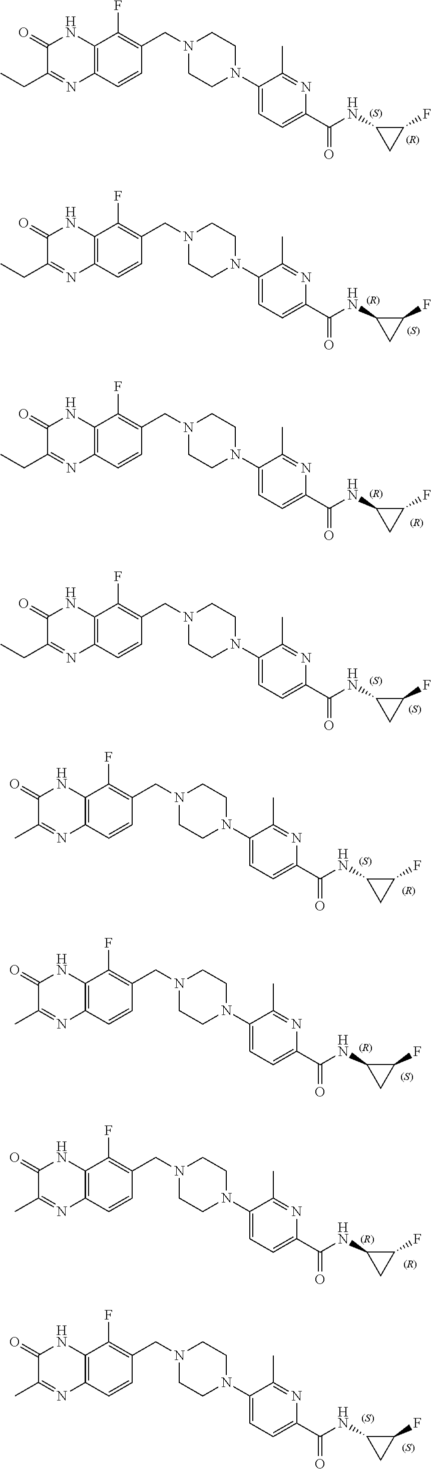

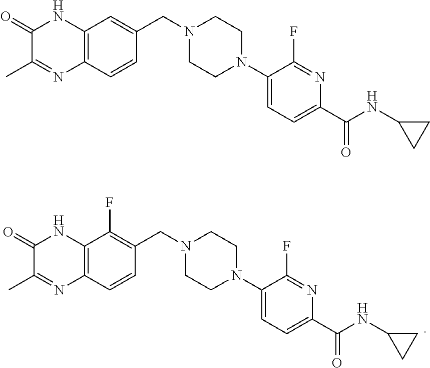

The present invention is further intended to provide a pharmaceutical composition comprising a pharmaceutically acceptable carrier, diluent, and/or excipient, as well as the compound of general formula (1) or the isomer thereof, the crystalline form thereof, the pharmaceutically acceptable salt thereof, the hydrate thereof, or the solvate thereof of the present invention as an active ingredient.

The present invention is still further intended to provide use of the compound of general formula (1) or the isomer thereof, the crystalline form thereof, the pharmaceutically acceptable salt thereof, the hydrate thereof, or the solvate thereof of the present invention or the pharmaceutical composition described above in preparing a medicament for treating, regulating, or preventing a disease related to PARP, wherein the disease is preferably cancer, and the cancer is a hematologic cancer or a solid tumor, wherein the cancer is preferably ovarian cancer, breast cancer, endometrial cancer, cervical cancer, pancreatic cancer, prostate cancer, non-small cell lung cancer, small cell lung cancer, esophageal cancer, colorectal cancer, renal cell carcinoma, bladder cancer, melanoma, or leukemia.

Still another objective of the present disclosure further provides a method for treating, regulating, or preventing a related disease mediated by PARP, which comprises administering to a subject a therapeutically effective amount of the compound of general formula (1) or the isomer thereof, the crystalline form thereof, the pharmaceutically acceptable salt thereof, the hydrate thereof, or the solvate thereof of the present disclosure, or the pharmaceutical composition described above.

It should be understood that both the aforementioned general description and the following detailed description of the present invention are exemplary and explanatory, and are intended to provide further explanation of the present invention claimed.

### BRIEF DESCRIPTION OF THE DRAWINGS

FIG. 1 shows the results of changes in tumor volume of Biological Example 5 of the present disclosure;

FIG. 2 shows the results of changes in body weight of mice of Biological Example 5 of the present disclosure.

### SYNTHESIS OF COMPOUNDS

Methods for preparing the compound of general formula (1) of the present disclosure are specifically described below, but these specific methods do not limit the present disclosure in any way.

The compound of general formula (1) described above may be synthesized using standard synthetic techniques or well-known techniques in combination with the methods described herein. In addition, the solvents, temperatures, and other reaction conditions mentioned herein may vary. Starting materials for the synthesis of the compounds may be obtained synthetically or commercially. The compounds described herein and other related compounds with different substituents may be synthesized using well-known techniques and starting materials, including the methods found in March, ADVANCED ORGANIC CHEMISTRY, 4^{th} Ed., (Wiley 1992); Carey and Sundberg, ADVANCED ORGANIC CHEMISTRY, 4^{th} Ed., Vols. A and B (Plenum 2000, 2001); and Green and Wuts, PROTECTIVE GROUPS IN ORGANIC SYNTHESIS, 3^{rd} Ed., (Wiley 1999). General methods for preparing the compounds may be altered by using appropriate reagents and conditions for introducing different groups into the molecular formulas provided herein.

In one aspect, the compounds described herein are prepared according to methods well known in the art. However, the conditions of the methods, such as reactants, solvents, bases, the amount of the compound used, reaction temperature, and time required for the reaction are not limited to the following explanation. The compounds of the present disclosure may also be conveniently prepared by optionally combining various synthetic methods described herein or known in the art, and such combinations may be easily determined by those skilled in the art to which the present disclosure pertains. In one aspect, the present disclosure further provides a method for preparing the compound of general formula (1), which is prepared using general reaction scheme 1 below:

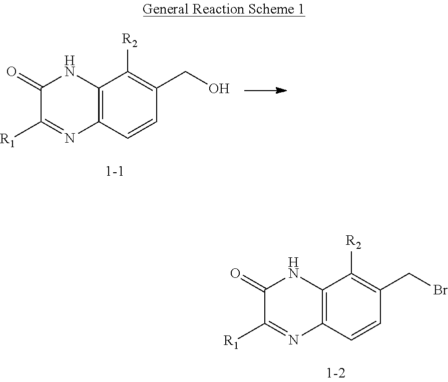

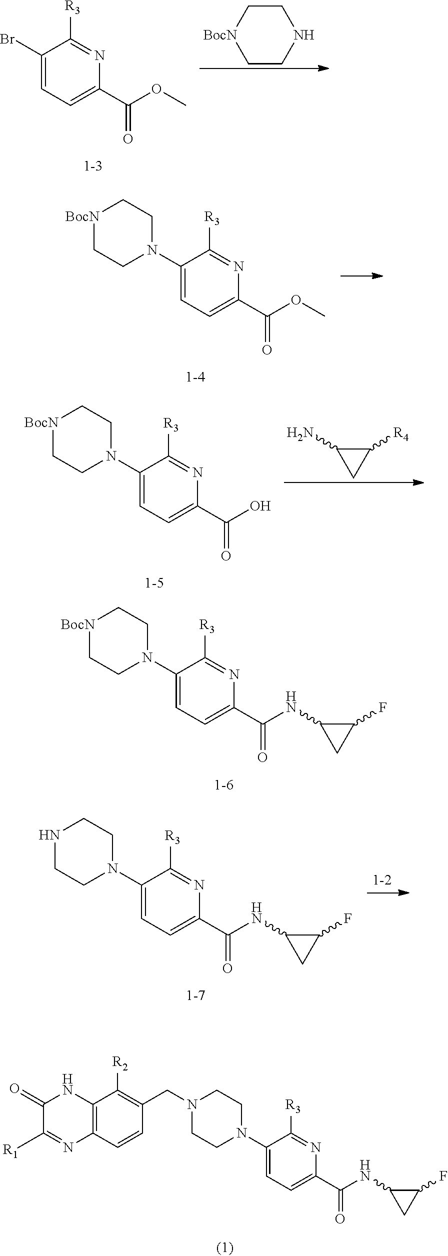

Embodiments of the compound of general formula (1) may be prepared according to general reaction scheme 1, wherein R₁, R₂, R₃, and R₄ are as defined above. As shown in general reaction scheme 1, compound 1-1 reacts with trimethylphosphine and carbon tetrabromide to generate compound 1-2, compound 1-3 undergoes a coupling reaction with N-Boc-piperazine to generate compound 1-4, compound 1-4 is hydrolyzed under alkaline conditions to generate compound 1-5, compound 1-5 condenses with the corresponding 2-fluorocyclopropanamine (single isomer or mixture) to generate compound 1-6, compound 1-6 is subjected to Boc removal under acidic conditions to generate compound 1-7, and compound 1-7 and compound 1-2 undergo a substitution reaction to generate the target compound of general formula (1).

### Further Forms of Compounds

“Pharmaceutically acceptable” herein refers to a substance, such as a carrier or diluent, which will not lead to loss of biological activity or properties of a compound and is relatively non-toxic. For example, when an individual is given a substance, the substance will not cause undesired biological effects or interact with any component contained therein in a deleterious manner.

The term “pharmaceutically acceptable salt” refers to a form of a compound that does not cause significant irritation to the organism receiving the administration or eliminate the biological activity and properties of the compound. In certain specific aspects, the pharmaceutically acceptable salt is obtained by subjecting the compound of the general formula to a reaction with acids or bases, wherein the acids or bases include, but are not limited to, those found in Stahl and Wermuth, Handbook of Pharmaceutical Salts: Properties, Selection, and Use, 1^{st} Ed., (Wiley, 2002).

It should be understood that references to pharmaceutically acceptable salts include solvent addition forms or crystalline forms, especially solvates or polymorphs. A solvate contains either stoichiometric or non-stoichiometric amount of solvent and is selectively formed during crystallization in a pharmaceutically acceptable solvent such as water and ethanol. Hydrates are formed when the solvent is water, or alcoholates are formed when the solvent is ethanol. The solvates of the compound of general formula (1) are conveniently prepared or formed according to the methods described herein. For example, hydrates of the compound of general formula (1) are conveniently prepared by recrystallization in a mixed solvent of water/organic solvent, wherein the organic solvent used includes, but is not limited to, tetrahydrofuran, acetone, ethanol, or methanol. Furthermore, the compounds described herein may be present in either a non-solvated form or a solvated form. In general, the solvated forms are considered equivalent to the non-solvated forms for purposes of the compounds and methods provided herein.

In other specific examples, the compound of general formula (1) is prepared in different forms including, but not limited to, amorphous, pulverized, and nanoparticle forms. In addition, the compound of general formula (1) includes crystalline forms, and may also be polymorphs. Polymorphs include different lattice arrangements of the same elements of a compound. Polymorphs generally have different X-ray diffraction spectra, infrared spectra, melting points, density, hardness, crystalline forms, optical and electrical properties, stability, and solubility. Different factors such as a recrystallization solvent, crystallization rate, and storage temperature may lead to a single dominant crystalline form.

In another aspect, the compound of general formula (1) may have a chiral center and/or axial chirality, and thus may be present in the form of a racemate, a racemic mixture, a single enantiomer, a diastereomeric compound, a single diastereomer, and a cis-trans isomer. Each chiral center or axial chirality will independently produce two optical isomers, and all possible optical isomers, diastereomeric mixtures, and pure or partially pure compounds are included within the scope of the present disclosure. The present disclosure is meant to include all such isomeric forms of these compounds.

The compound of the present disclosure may contain unnatural proportions of atomic isotopes at one or more of the atoms that constitute the compound. For example, the compound may be labeled with radioactive isotopes, such as tritium (H), iodine-125 (¹²¹I), and C-14 (¹⁴C). For another example, deuterium can be used to substitute a hydrogen atom to form a deuterated compound. The bond formed by deuterium and carbon is stronger than that formed by ordinary hydrogen and carbon. Compared with an undeuterated medicament, the deuterated medicament generally has the advantages of reduced toxic and side effects, increased pharmaceutical stability, enhanced efficacy, prolonged pharmaceutical in vivo half-life, and the like. All isotopic variations of the compound of the present disclosure, whether radioactive or not, are contained within the scope of the present disclosure.

Any atom of the compound of the present disclosure, unless otherwise specified, refers to an isotope of the atom in stable state of the compound. Unless otherwise specified, when a site in a molecular structure is selected as “H” or “hydrogen”, the site should be understood as having the natural abundance of the hydrogen isotope. Similarly, unless otherwise specified, when a site is selected as “D” or “deuterium”, the site should be understood to have a deuterium isotopic abundance that is at least 3000 times its natural abundance (the natural abundance of the deuterium isotope is 0.015%).

More preferably, each deuterated site of the deuterated compounds of the present disclosure has a deuterium atom abundance that is at least 3500 times its natural abundance (52.2% deuterium atom enrichment). More preferably, the deuterium atom abundance is at least 4500 times the natural abundance (67.5% deuterium atom enrichment). More preferably, the deuterium atom abundance is at least 5000 times the natural abundance (75% deuterium atom enrichment). More preferably, the deuterium atom abundance is at least 6000 times the natural abundance (90% deuterium atom enrichment). More preferably, the deuterium atom abundance is at least 6333 times the natural abundance (95% deuterium atom enrichment). More preferably, the deuterium atom abundance is at least 6466.7 times the natural abundance (97% deuterium atom enrichment). More preferably, the deuterium atom abundance is at least 6600 times the natural abundance (99% deuterium atom enrichment). More preferably, the deuterium atom abundance is at least 6633.3 times the natural abundance (99.5% deuterium atom enrichment).

### Terminology

Unless otherwise stated, the terms used in the present application, including those in the specification and claims, are defined as follows. It must be noted that in the specification and the appended claims, the singular forms “a” and “an” include plural meanings unless clearly indicated otherwise. Unless otherwise stated, conventional methods for mass spectrometry, nuclear magnetic resonance spectroscopy, HPLC, protein chemistry, biochemistry, recombinant DNA techniques, and pharmacology are used. As used herein, “or” or “and” refers to “and/or” unless otherwise stated.

Unless otherwise specified, “alkyl” refers to a saturated aliphatic hydrocarbon group, including linear and branched groups containing 1 to 6 carbon atoms. Lower alkyl groups containing 1 to 4 carbon atoms, such as methyl, ethyl, propyl, 2-propyl, n-butyl, isobutyl, and tert-butyl, are preferred. Lower alkyl groups containing 1 to 3 carbon atoms, such as methyl, ethyl, propyl, and 2-propyl, are further preferred. As used herein, “alkyl” includes unsubstituted and substituted alkyl, particularly alkyl substituted with one or more halogens. Preferred alkyl is selected from CH₃, CH₃CH₂, CF₃, CHF₂, CF₃CH₂, CF₃(CH₃)CH, ⁱPr, ⁿPr, ⁱBu, ⁿBu, and ^{t}Bu.

Unless otherwise specified, “cycloalkyl” refers to a non-aromatic hydrocarbon ring system (monocyclic, bicyclic, or polycyclic), and is preferably a non-aromatic hydrocarbon ring system containing 3 to 14 ring carbon atoms (C₃₋₁₄ cycloalkyl). In some embodiments, cycloalkyl has 3 to 10 ring carbon atoms (C₃₋₁₀ cycloalkyl). In some embodiments, cycloalkyl has 3 to 8 ring carbon atoms (C₃₋₈ cycloalkyl). In some embodiments, cycloalkyl has 3 to 7 ring carbon atoms (C₃₇ cycloalkyl). In some embodiments, cycloalkyl has 3 to 6 ring carbon atoms (C₃₋₆ cycloalkyl). In some embodiments, cycloalkyl has 4 to 6 ring carbon atoms (C₄₋₆ cycloalkyl). In some embodiments, cycloalkyl has 5 to 6 ring carbon atoms (C₅₋₆ cycloalkyl). In some embodiments, cycloalkyl has 5 to 10 ring carbon atoms (C₅₋₁₀ cycloalkyl). For cycloalkyl, partially unsaturated cycloalkyl may be referred to as “cycloalkenyl” if the carbocyclic ring contains at least one double bond, or “cycloalkynyl” if the carbocyclic ring contains at least one triple bond. Cycloalkyl may include monocyclic or polycyclic groups (e.g., having 2, 3, or 4 fused rings) and spiro rings. In some embodiments, cycloalkyl is monocyclic. In some embodiments, cycloalkyl is bicyclic. In some embodiments, cycloalkyl is monocyclic or bicyclic. In some embodiments, cycloalkyl is tricyclic. The ring carbon atoms of cycloalkyl may optionally be oxidized to form an oxo or thio group. Cycloalkyl further includes cycloalkylene. In some embodiments, cycloalkyl contains 0, 1, or 2 double bonds. In some embodiments, cycloalkyl contains 1 or 2 double bonds (partially unsaturated cycloalkyl). In some embodiments, cycloalkyl may be fused to aryl, heteroaryl, cycloalkyl, and heterocycloalkyl. In some embodiments, cycloalkyl may be fused to aryl, cycloalkyl, and heterocycloalkyl. In some embodiments, cycloalkyl may be fused to aryl and heterocycloalkyl. In some embodiments, cycloalkyl may be fused to aryl and cycloalkyl. Examples of cycloalkyl include cyclopropyl, cyclobutyl, cyclopentyl, cyclohexyl, cycloheptyl, cyclopentenyl, cyclohexenyl, cyclohexadienyl, cycloheptatrienyl, norcamphanyl, norpinanyl, norcarnyl, bicyclo[1.1.1]pentyl, bicyclo[2.1.1]hexyl, and the like.

Unless otherwise specified, “halogen” (or halo) refers to fluorine, chlorine, bromine, or iodine. The term “halo” (or “halogenated”) before a group name indicates that the group is partially or fully halogenated, that is, substituted in any combination with F, Cl, Br, or I, preferably with F or Cl.

Unless otherwise specified, the term “substituted” means that one or more hydrogen atoms on a designated atom or group are substituted with one or more substituents other than hydrogen without exceeding the normal valence of the designated atom. For example, one or more hydrogens of alkyl, alkylene, alkenyl, alkynyl, hydroxy, amino, or the like may be substituted with one or more substituents. The substituents include, but are not limited to, alkyl, alkenyl, alkynyl, alkoxy, acyl, amino, amido, amidino, aryl, azido, carbamoyl, carboxy, carboxylate, cyano, guanidino, halogen, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. The definition of “substituted” does not include analogous indeterminate structures obtained by defining substituents having further substituents attached to infinity (e.g., substituted aryl having substituted alkyl is itself substituted with substituted aryl, which is further substituted with substituted heteroalkyl, and the like). Unless otherwise specified, the maximum number of consecutive substitutions in the compound described herein is three. For example, the consecutive substitutions of substituted aryl with two other substituted aryls are limited to ((substituted aryl)substituted aryl)substituted aryl. Similarly, the definitions described above do not include impermissible substitution patterns (e.g., methyl substituted with 5 fluorines or heteroaryl having two adjacent oxygen ring atoms). Such impermissible substitution patterns are well known to those skilled in the art. When used to modify a chemical group, “substituted” may describe other chemical groups as defined herein. For example, the term “substituted aryl” includes, but is not limited to, “alkylaryl”. Unless otherwise specified, if a group is described as optionally substituted, any substituent of the group is itself unsubstituted.

“Optional” or “optionally” means that the subsequently described event or circumstance may, but does not necessarily, occur, and the description includes instances where the event or circumstance occurs and instances where it does not.

Unless otherwise specified, it will be understood that the word “comprise” or variations thereof such as “comprises” or “comprising” refers to the inclusion of a stated element or integer or a group of elements or integers, but not the exclusion of any other element or integer or a group of elements or integers.

The term “membered ring” includes any cyclic structure. The term “membered” is intended to refer to the number of backbone atoms that form a ring. For example, cyclohexyl, pyridinyl, pyranyl, and thiopyranyl are six-membered rings, and cyclopentyl, pyrrolyl, furanyl, and thienyl are five-membered rings.

The term “moiety” refers to a specific portion or functional group of a molecule. A chemical moiety is generally considered to be a chemical entity contained in or attached to a molecule.

The term “isomer” refers to any tautomer, stereoisomer, atropisomer, isotopic isomer, enantiomer, or diastereomer of any compound of the present disclosure. The compound of the present disclosure may have one or more chiral centers or double bonds, and thus exists in the form of stereoisomers, e.g., double bond isomers (i.e., E/Z geometric isomers), or diastereomers (e.g., enantiomers (i.e., (+) or (−)) or cis/trans isomers). The compound of the present disclosure therefore encompasses all corresponding stereoisomers, i.e., stereoisomerically pure (e.g., geometrically pure, enantiomerically pure, or diastereomerically pure) forms, as well as mixtures of enantiomers and stereoisomers, e.g., racemates. The mixtures of enantiomers and stereoisomers of the compound of the present disclosure may be resolved into their component enantiomers or stereoisomers by well-known methods, such as chiral gas chromatography and chiral high-performance liquid chromatography, and by crystallization of the compound in the form of chiral salt complexes or in chiral solvents. Enantiomers and stereoisomers may also be obtained from stereomerically pure or enantiomerically pure intermediates, reagents, and catalysts by well-known asymmetric synthetic methods.

The term “isotopic isomer” refers to distinct molecules that differ in structure only by their isotopic composition and are identical in the remaining structure.

Unless otherwise stated, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ), or a wavy line ( [symbol] ) represents a straight solid bond ( [symbol] ) or a straight dashed bond ( [symbol] ).

Unless otherwise stated, a single bond or a double bond is represented by [symbol] .

### Specific Pharmaceutical and Medical Terminology

The term “acceptable”, as used herein, means that a formula component or an active ingredient does not unduly and adversely affect a general therapeutic target's health.

The terms “treatment”, “treatment course”, and “therapy”, as used herein, include alleviating, inhibiting, or ameliorating a symptom or condition of a disease; inhibiting the development of complications; ameliorating or preventing underlying metabolic syndrome; inhibiting the development of a disease or symptom, e.g., controlling the progression of a disease or condition; alleviating a disease or symptom; leading to disease or symptom regression; and alleviating a complication caused by a disease or symptom, or preventing or treating a sign caused by a disease or symptom. As used herein, a compound or pharmaceutical composition, when administered, can ameliorate a disease, symptom, or condition, which particularly refers to ameliorating the severity, delaying the onset, slowing the progression, or reducing the duration of the disease. Fixed or temporary administration, or continuous or intermittent administration, may be attributed to or associated with the administration.

“Active ingredient” refers to the compound of general formula (1), and pharmaceutically acceptable inorganic or organic salts of the compound of general formula (1). The compound of the present disclosure may contain one or more asymmetric centers (chiral center or axial chirality) and thus occurs in the forms of a racemate, a racemic mixture, a single enantiomer, a diastereomeric compound, and a single diastereomer. Asymmetric centers that may be present depend on the nature of the various substituents on the molecule. Each of such asymmetric centers will independently produce two optical isomers, and all possible optical isomers, diastereomeric mixtures and pure or partially pure compounds are included within the scope of the present disclosure. The present disclosure is meant to include all such isomeric forms of these compounds. The terms such as “compound”, “composition”, “agent”, or “medicine or medicament” are used interchangeably herein and all refer to a compound or composition that, when administered to an individual (human or animal), is capable of inducing a desired pharmacological and/or physiological response by local and/or systemic action.

The term “administered, administering, or administration” refers herein to the direct administration of the compound or composition, or the administration of a prodrug, derivative, analog, or the like of the active compound.

Although the numerical ranges and parameters defining the broad scope of the present disclosure are approximations, the related numerical values set forth in the specific examples have been presented herein as precisely as possible. Any numerical value, however, inherently contains a standard deviation necessarily resulting from certain methods of testing. Herein, “about” generally means that the actual numerical value is within a particular numerical value or range ±10%, 5%, 1%, or 0.5%. Alternatively, the term “about” indicates that the actual numerical value falls within the acceptable standard error of a mean, as considered by those skilled in the art. All ranges, quantities, numerical values, and percentages used herein (e.g., to describe an amount of a material, a length of time, a temperature, an operating condition, a quantitative ratio, and the like) are to be understood as being modified by the word “about”, except in the experimental examples or where otherwise explicitly indicated. Accordingly, unless otherwise contrarily stated, the numerical parameters set forth in the specification and the appended claims are all approximations that may vary as desired. At least, these numerical parameters should be understood as the significant digits indicated or the numerical values obtained using conventional rounding rules.

Unless otherwise defined in the specification, the scientific and technical terms used herein have the same meaning as commonly understood by those skilled in the art. Furthermore, nouns in their singular forms used in the specification encompass their plural forms, unless contradicted by context; nouns in their plural forms used also encompass their singular forms.

### Therapeutic Use

The present disclosure provides a method for treating a disease, including but not limited to a related condition involving PARP activity (e.g., cancer), with the compound of general formula (1) or the pharmaceutical compositions of the present disclosure.

In some embodiments, a method for treating cancer is provided, the method comprising administering to an individual in need thereof an effective amount of any of the aforementioned pharmaceutical compositions comprising the compound of structural general formula (1). In some embodiments, the cancer is mediated by PARR In other embodiments, the cancer is a hematologic cancer and a solid tumor, including but not limited to leukemia, breast cancer, lung cancer, pancreatic cancer, colon cancer, colorectal cancer, bladder cancer, brain cancer, peritoneal cancer, urothelial carcinoma, prostate cancer, liver cancer, fallopian tube cancer, ovarian cancer, endometrial cancer, cervical cancer, renal cell carcinoma, colorectal cancer, head and neck cancer, esophageal cancer, gastric cancer, melanoma, glioblastoma, Ewing's sarcoma, soft tissue sarcoma, mesothelioma, or all cancer metastases. The cancer is preferably ovarian cancer, breast cancer, endometrial cancer, cervical cancer, pancreatic cancer, prostate cancer, non-small cell lung cancer, small cell lung cancer, esophageal cancer, colorectal cancer, renal cell carcinoma, bladder cancer, melanoma, or leukemia.

### Route of Administration

The compound and the pharmaceutically acceptable salt thereof of the present disclosure can be made into various formulations comprising a safe and effective amount of the compound or the pharmaceutically acceptable salt thereof of the present disclosure, and a pharmaceutically acceptable excipient or carrier. The “safe and effective amount” means that the amount of the compound is sufficient to significantly improve the condition without causing serious side effects. The safe and effective amount of the compound is determined according to the age, condition, course of treatment, and other specific conditions of a treated subject.

The “pharmaceutically acceptable excipient or carrier” refers to one or more compatible solid or liquid fillers or gel substances that are suitable for human use and must be of sufficient purity and sufficiently low toxicity. “Compatible” herein means that the components of the composition are capable of intermixing with the compound of the present disclosure and with each other, without significantly diminishing the pharmaceutical efficacy of the compound. Examples of pharmaceutically acceptable excipients or carriers include cellulose and derivatives thereof (e.g., sodium carboxymethylcellulose, sodium ethylcellulose, or cellulose acetate), gelatin, talc, solid lubricants (e.g., stearic acid or magnesium stearate), calcium sulfate, vegetable oil (e.g., soybean oil, sesame oil, peanut oil, or olive oil), polyols (e.g., propylene glycol, glycerol, mannitol, or sorbitol), emulsifiers (e.g., Tween®), wetting agents (e.g., sodium lauryl sulfate), colorants, flavoring agents, stabilizers, antioxidants, preservatives, pyrogen-free water, etc.

When the compound of the present disclosure is administered, it may be administered orally, rectally, parenterally (intravenously, intramuscularly, or subcutaneously), or topically.

Solid dosage forms for oral administration include capsules, tablets, pills, pulvises, and granules. In these solid dosage forms, the active compound is mixed with at least one conventional inert excipient (or carrier), such as sodium citrate or dicalcium phosphate, or with the following ingredients: (a) fillers or extenders, such as starch, lactose, sucrose, glucose, mannitol, and silicic acid; (b) binders, such as hydroxymethyl cellulose, alginate, gelatin, polyvinylpyrrolidone, sucrose, and acacia; (c) humectants, such as glycerol; (d) disintegrants, such as agar, calcium carbonate, potato or tapioca starch, alginic acid, certain complex silicates, and sodium carbonate; (e) solution retarders, such as paraffin; (f) absorption accelerators, such as quaternary ammonium compounds; (g) wetting agents, such as cetyl alcohol and glycerol monostearate; (h) adsorbents, such as kaolin; and (i) lubricants, such as talc, calcium stearate, magnesium stearate, solid polyethylene glycol and sodium lauryl sulfate, or mixtures thereof. In the case of capsules, tablets, and pills, the dosage forms may further include buffers.

Solid dosage forms such as tablets, dragees, capsules, pills, and granules can be prepared using coatings and shells such as enteric coatings and other materials well known in the art. They may include opacifying agents, and the active compound or compound in such a composition may be released in a certain part of the digestive tract in a delayed manner. Examples of embedding components that can be used are polymeric substances and wax-based substances. If necessary, the active compound can also be in microcapsule form with one or more of the excipients described above.

Liquid dosage forms for oral administration include pharmaceutically acceptable emulsions, solutions, suspensions, syrups, and elixirs. In addition to the active compound, the liquid dosage form may include inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide, and oils, especially cottonseed oil, peanut oil, corn germ oil, olive oil, castor oil and sesame oil, or mixtures of these substances.

Besides such inert diluents, the composition may further include adjuvants, such as wetting agents, emulsifiers, suspending agents, sweeteners, flavoring agents, and perfuming agents.

In addition to the active compound, suspensions may include suspending agents, such as ethoxylated isostearyl alcohols, polyoxyethylene sorbitol and sorbitan esters, microcrystalline cellulose, aluminum methylate and agar, or mixtures of these substances.

Compositions for parenteral injection may include physiologically acceptable sterile aqueous or anhydrous solutions, dispersions, suspensions, or emulsions, and sterile powders for redissolving into sterile injectable solutions or dispersions. Suitable aqueous and non-aqueous carriers, diluents, solvents, or excipients include water, ethanol, polyols, and suitable mixtures thereof.

Dosage forms for topical administration of the compound of the present disclosure include ointments, pulvises, patches, sprays, and inhalants. The active ingredient is mixed under sterile conditions with a physiologically acceptable carrier and any preservatives, buffers, or propellants that may be required if necessary.

The compound of the present disclosure may be administered alone or in combination with other pharmaceutically acceptable compounds. When the pharmaceutical composition is used, a safe and effective amount of the compound of the present disclosure is administered to a mammal (such as a human) to be treated, wherein the dose of administration is a pharmaceutically effective dose. For a human of 60 kg, the daily dose of administration is usually 1-2000 mg, preferably 50-1000 mg. In determining a specific dose, such factors as the route of administration, the health condition of the patient, and the like will also be considered, which are well-known to skilled physicians.

The above features mentioned in the present disclosure or those mentioned in the examples may be combined arbitrarily. All the features disclosed in this specification may be used with any composition form and the various features disclosed in this specification may be replaced with any alternative features that provide the same, equivalent, or similar purpose. Thus, unless otherwise specified, the features disclosed herein are merely general examples of equivalent or similar features.

### DETAILED DESCRIPTION

Various specific aspects, features, and advantages of the compounds, methods, and pharmaceutical compositions described above will be set forth in detail in the following description, which will make the content of the present disclosure very clear. It should be understood that the detailed description and examples below describe specific examples for reference only. After reading the description of the present disclosure, those skilled in the art can make various changes or modifications to the present disclosure, and such equivalents also fall within the scope of the present application defined herein.

In all the examples, ¹H-NMR spectra were recorded with a Varian Mercury 400 nuclear magnetic resonance spectrometer, and chemical shifts are represented by δ (ppm); silica gel for separation was 200-300 mesh silica gel if not specified, and the ratio of the eluents was a volume ratio.

### Example 1. Synthesis of Compound 1

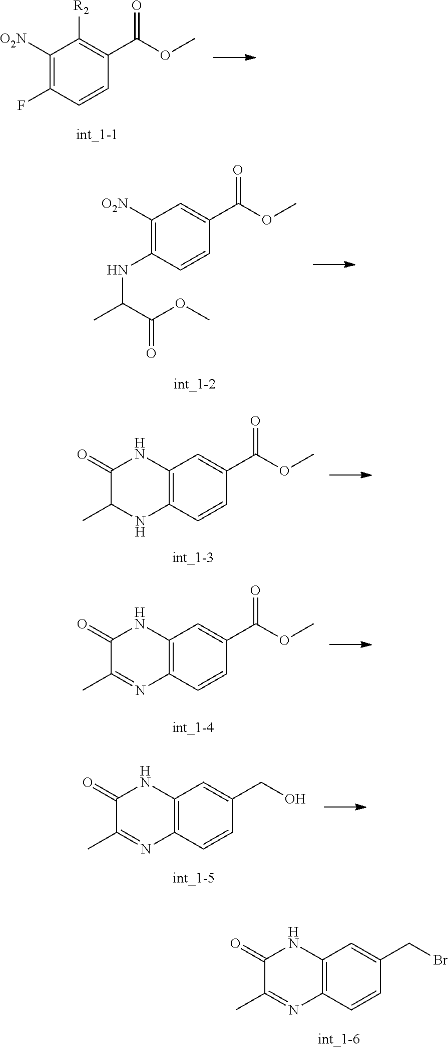

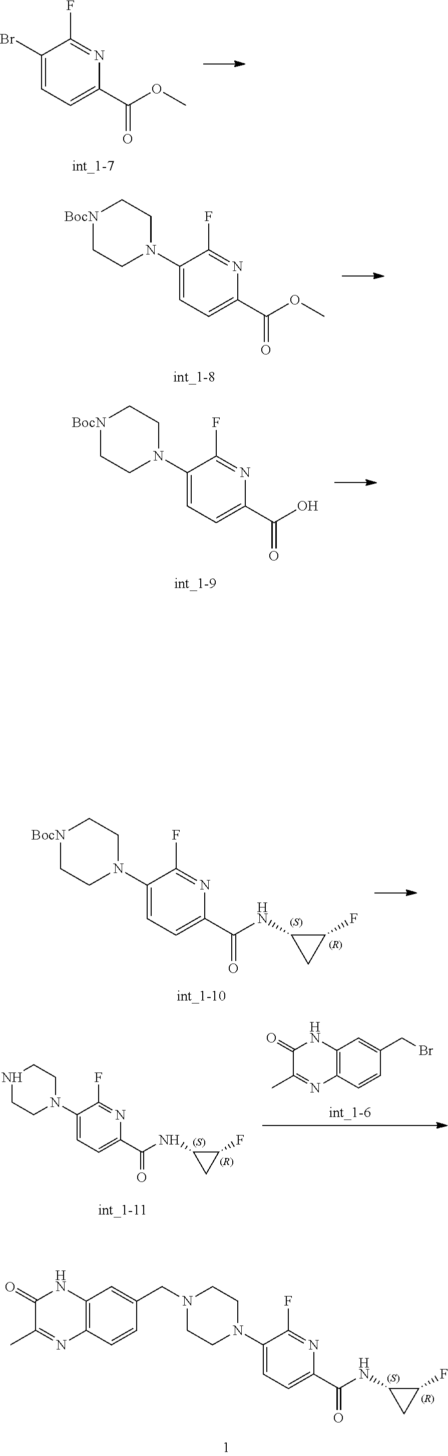

### Step 1: Synthesis of Compound Int_1-2:

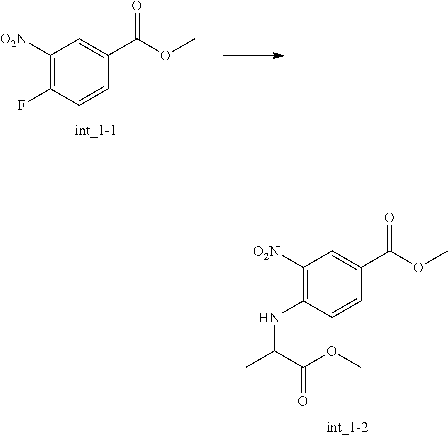 [SMILES: COC(=O)c1ccc(F)c([N+](=O)[O-])c1 ; COC(=O)c1ccc(NC(C)C(=O)OC)c([N+](=O)[O-])c1]

Methyl 4-fluoro-3-nitrobenzoate (15.00 g, 75.32 mmol) and DL-alanine methyl ester hydrochloride (10.51 g, 75.32 mmol) were added to a reaction flask and dissolved in anhydrous N,N-dimethylformamide (150 mL), and diisopropylethylamine (29.21 g, 225.97 mmol) was slowly added dropwise. The reaction solution was stirred at 25° C. for 12 h under nitrogen atmosphere. The reaction solution was poured into water (500 mL), and the mixture was extracted with ethyl acetate (300 mL×3). The organic layers were combined, washed with saturated brine (500 mL×3), dried over anhydrous sodium sulfate, and filtered. The filtrate was concentrated to give a crude product. The crude product was purified by slurrying with dichloromethane (25 mL) to give intermediate int_1-2.

LCMS m/z (ESI): 283 [M+H]⁺.

### Step 2: Synthesis of Compound Int_1-3:

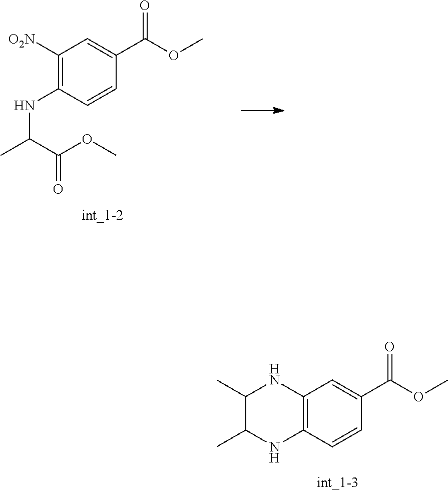

Compound int_1-2 (7.90 g, 28.00 mmol) was dissolved in glacial acetic acid (80 mL), and iron powder (15.68 g, 280.00 mmol) was added. The reaction solution was stirred at 50° C. for 3 h. The reaction solution was diluted with water (80 mL) and subjected to suction filtration while hot. The filter cake was rinsed with methanol/dichloromethane (1:10, 50 mL×3), and the filtrate was extracted with methanol/dichloromethane (1:10, 100 mL×3) and concentrated to dryness under reduced pressure to give crude compound int_1-3 as a gray solid.

LCMS m/z (ESI): 221.1 [M+H]⁺.

### Step 3: Synthesis of Compound Int_1-4:

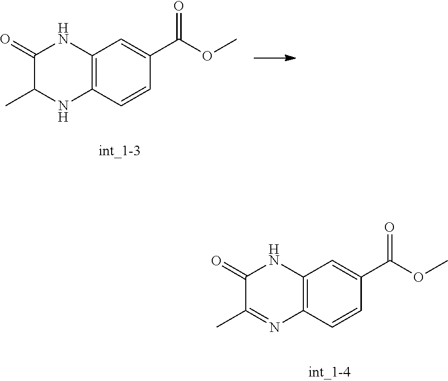 [SMILES: COC(=O)c1ccc2c(c1)NC(=O)C(C)N2 ; COC(=O)c1ccc2nc(C)c(=O)[nH]c2c1]

Compound int_1-3 (6.5 g) was dissolved in dichloromethane (70 mL), and 2,3-dichloro-5,6-dicyanobenzoquinone (7.63 g, 33.60 mmol) was added. The reaction solution was stirred at 25° C. for 3 h. The reaction solution was concentrated under reduced pressure, and the residue was slurried with a saturated aqueous sodium bicarbonate solution (100 mL) and subjected to suction filtration. The filter cake was rinsed with a saturated aqueous sodium bicarbonate solution (50 mL×3), collected, and dried to give compound int_1-4 as a brown solid.

LCMS m/z (ESI): 219.0 [M+H]⁺.

### Step 4: Synthesis of Compound Int_1-5:

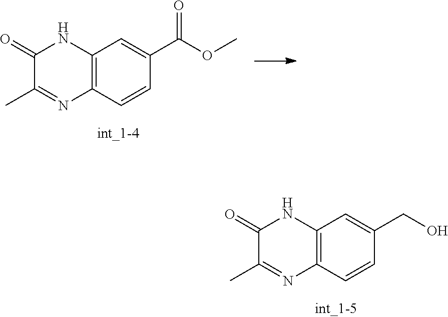 [SMILES: COC(=O)c1ccc2nc(C)c(=O)[nH]c2c1 ; Cc1nc2ccc(CO)cc2[nH]c1=O]

Compound int_1-4 (8.65 g) was dissolved in anhydrous tetrahydrofuran (90 mL), and the solution was thoroughly purged with nitrogen. The reaction solution was cooled to 0° C., and a solution of lithium aluminum hydride in tetrahydrofuran (15.8 mL, 39.5 mmol, 2.5 M) was added dropwise. The reaction solution was stirred at 0° C. for 2 h. Sodium sulfate decahydrate was added to the reaction solution with stirring at 0° C. until no foam generation was observed, indicating complete quenching. The system was subjected to suction filtration, and the filter cake was rinsed with methanol/dichloromethane (1:10, 50 mL×3). The filtrate was concentrated under reduced pressure to give a crude product. The crude product was separated and purified by silica gel chromatography to give intermediate int_1-5.

LCMS m/z (ESI): 191.1 [M+H]⁺.

### Step 5: Synthesis of Compound Int_1-6:

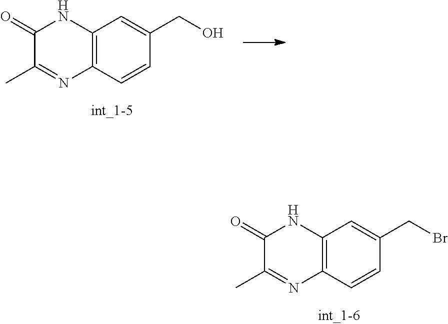

Compound int_1-5 (400 mg, 2.10 mmol) was dissolved in anhydrous dichloromethane (8 mL), and carbon tetrabromide (382.7 mg, 4.21 mmol) was added. The mixture was purged with nitrogen, and a solution of trimethylphosphine in tetrahydrofuran (8.42 mL, 8.42 mmol, 1 M) was added dropwise at 0° C. After the dropwise addition was completed, the reaction solution was slowly warmed to room temperature and stirred for 3 h. The reaction solution was concentrated under reduced pressure to give a crude product. The crude product was separated and purified by silica gel chromatography to give intermediate int_1-6.

### Step 6: Synthesis of Compound Int_1-8:

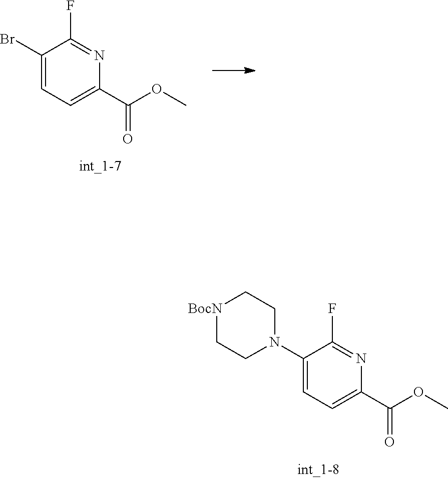 [SMILES: COC(=O)c1ccc(Br)c(F)n1 ; COC(=O)c1ccc(N2CCN(C(=O)OC(C)(C)C)CC2)c(F)n1]

Methyl 5-bromo-6-fluoropicolinate (500 mg, 2.14 mmol) and N-Boc-piperazine (437.73 mg, 2.35 mmol) were dissolved in methylbenzene (20 mL), and cesium carbonate (2.09 g, 6.41 mmol) was added. S-(+)-1,1-binaphthyl-2,2-diphemyl phosphine (133.04 mg, 213.65 μmol) and palladium(II) acetate (47.97 mg, 213.65 μmol) were then added to the reaction solution. The system was purged 3 times with nitrogen. The reaction solution was stirred at 80° C. for 16 h under nitrogen atmosphere. After cooling, water (30 mL) was added to the reaction solution, and the mixed solution was extracted with ethyl acetate (30 mL×3). The organic phases were combined, dried over anhydrous sodium sulfate, and subjected to suction filtration, and the filtrate was concentrated to dryness under reduced pressure. The residue was then separated and purified by silica gel chromatography to give intermediate int_1-8.

LCMS m/z (ESI): 284.0 [M−tBu+H]⁺.

### Step 7: Synthesis of Compound Int_1-9:

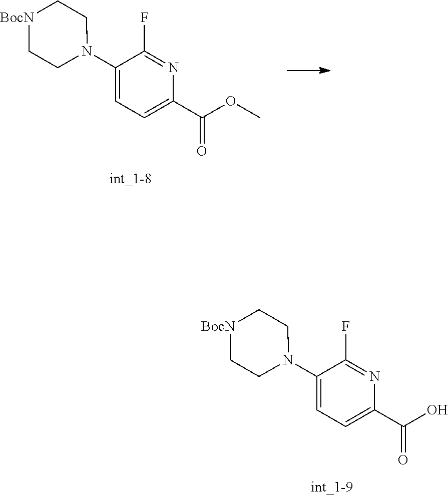 [SMILES: COC(=O)c1ccc(N2CCN(C(=O)OC(C)(C)C)CC2)c(F)n1 ; CC(C)(C)OC(=O)N1CCN(c2ccc(C(=O)O)nc2F)CC1]

Compound int_1-8 (565 mg, 1.66 mmol) was dissolved in methanol (5 mL) and tetrahydrofuran (5 mL), and a solution of sodium hydroxide (332.95 mg, 8.32 mmol) in water (5 mL) was added. The reaction solution was stirred at 20° C. for 12 h. The reaction solution was concentrated under reduced pressure, and water (20 mL) was added. The mixture was adjusted to pH 3-4 with 2 M diluted hydrochloric acid and extracted with ethyl acetate (20 mL×3). The organic phases were combined, washed with saturated brine (30 mL×2), dried over anhydrous sodium sulfate, and filtered. The filtrate was concentrated to give compound int_1-9 as a white solid.

### Step 8: Synthesis of Compound Int_1-10:

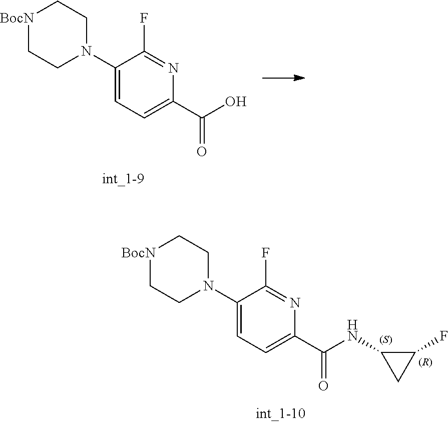

Compound int_1-9 (300 mg, 0.92 mmol), N,N,N′,N′-tetramethyl-O-(7-azabenzotriazol-1-yl)uronium hexafluorophosphate (420 mg, 1.11 mmol), and (1S-cis)-2-fluorocyclopropanamine (103 mg, 1.38 mmol) were dissolved in N,N-dimethylformamide (3 mL), and diisopropylethylamine (357 mg, 2.77 mmol) was added. The reaction solution was stirred at 25° C. for 2 h. Water (30 mL) was added to the reaction solution, and the mixture was extracted with ethyl acetate (30 mL×3). The organic phases were combined, washed with saturated brine (30 mL×3), dried over anhydrous sodium sulfate, and subjected to suction filtration, and the filtrate was concentrated to dryness under reduced pressure. The residue was separated and purified by silica gel chromatography to give intermediate int_1-10.

LCMS m/z (ESI): 383.2 [M+H]⁺.

### Step 9: Synthesis of Compound Int_1-11:

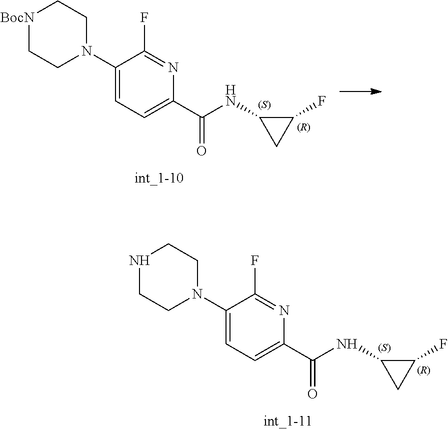

Compound int_1-10 (350 mg, 0.92 mmol) was dissolved in dichloromethane (10 mL), and a solution of hydrogen chloride in dioxane (10 mL, 4 M) was added. The reaction solution was stirred at 25° C. for 1 h. The reaction solution was concentrated to dryness under reduced pressure to give crude compound int_1-11 as a yellow oil.

LC-MS m/z (ESI): 283.1 [M+H]⁺.

### Step 10: Synthesis of Compound 1:

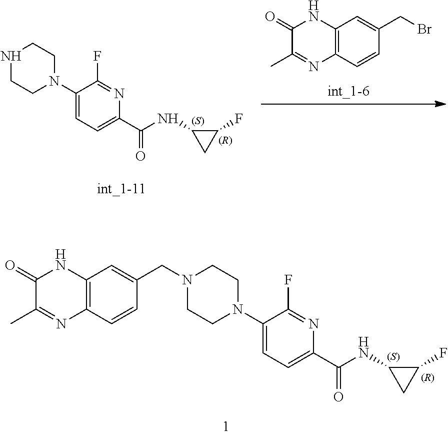

Compound int_1-11 (280 mg), int_1-6 (251 mg, 0.99 mmol), and anhydrous potassium carbonate (411 mg, 2.98 mmol) were dissolved in anhydrous acetonitrile (50 mL), and the mixture was stirred at 60° C. for 4 h. After cooling, water (200 mL) was added to the reaction solution, and the mixture was extracted with ethyl acetate (30 mL×3). The organic phases were combined, washed with saturated brine (20 mL×3), dried over anhydrous sodium sulfate, and subjected to suction filtration. The filtrate was concentrated to dryness under reduced pressure, and the residue was then purified by C18 preparative liquid chromatography (formic acid system) to give compound 1 (formate) as a yellow solid.

LCMS m/z (ESI): 455.2 [M+H]⁺.

¹H NMR (400 MHz, DMSO-d₆) δ 12.26 (s, 1H), 8.36 (d, J=4.4 Hz, 1H), 8.16 (s, 1H), 7.86 (d, J=7.9 Hz, 1H), 7.71-7.50 (m, 2H), 7.33-7.15 (m, 2H), 4.85-4.64 (m, 1H), 3.61 (s, 2H), 3.18 (m, 4H), 2.82 (m, 1H), 2.56 (m, 4H), 2.39 (s, 3H), 1.33 (m, 1H), 1.07 (m, 1H).

### Example 2. Synthesis of Compound 2

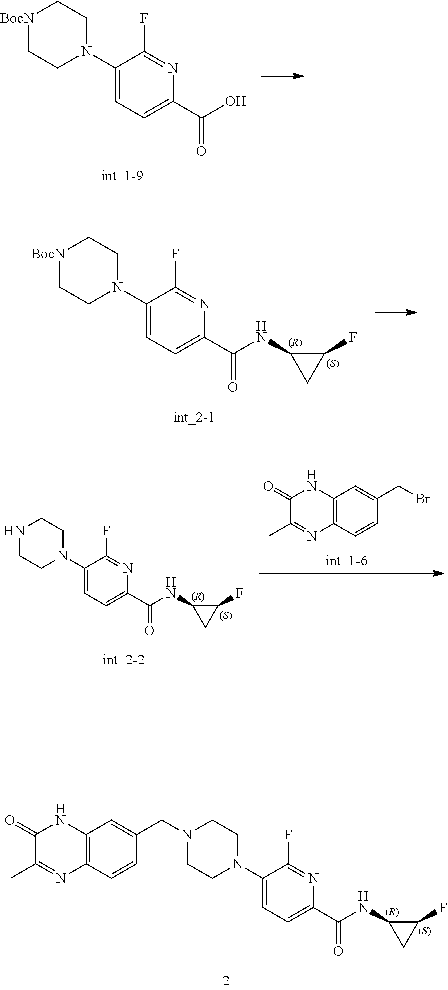

### Step 1: Synthesis of Compound Int_2-1:

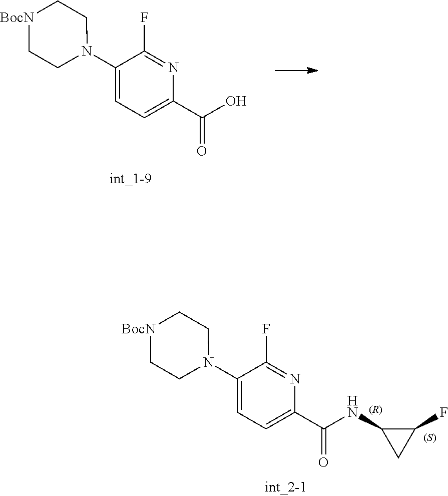

Compound int_1-9 (600 mg, 1.84 mmol) was dissolved in N,N-dimethylformamide (10 mL), and (1R,2S)-2-fluorocyclopropanamine 4-methylbenzenesulfonate (507 mg, 2.21 mmol) was added, followed by addition of N,N,N′,N′-tetramethyl-O-(7-azabenzotriazol-1-yl)uronium hexafluorophosphate (908 mg, 2.39 mmol) and N,N-diisopropylethylamine (949 mg, 7.36 mmol). The reaction solution was stirred at room temperature for 16 h. Water (10 mL) was added to the reaction solution, and the mixture was extracted with ethyl acetate (50 mL×3). The organic phases were combined, dried over anhydrous sodium sulfate, and subjected to suction filtration, and the filtrate was concentrated to dryness under reduced pressure. The residue was then separated and purified by silica gel chromatography to give intermediate int_2-1.

LCMS m/z (ESI): 383.2 [M+H]⁺.

### Step 2: Synthesis of Compound Int_2-2:

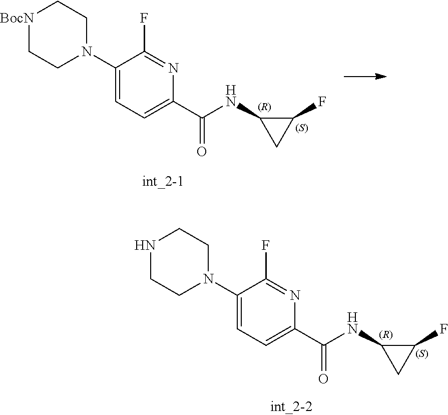

Compound int_2-1 (340 mg, 0.889 mmol) was dissolved in dichloromethane (10 mL), and a solution of hydrogen chloride in dioxane (5 mL, 4 M) was added. The reaction solution was stirred at room temperature for 2 h. The reaction solution was concentrated to dryness under reduced pressure to give crude compound int_2-2 as a reddish-brown solid.

LCMS m/z (ESI): 283.1 [M+H]⁺.

Step 3: Synthesis of compound 2:

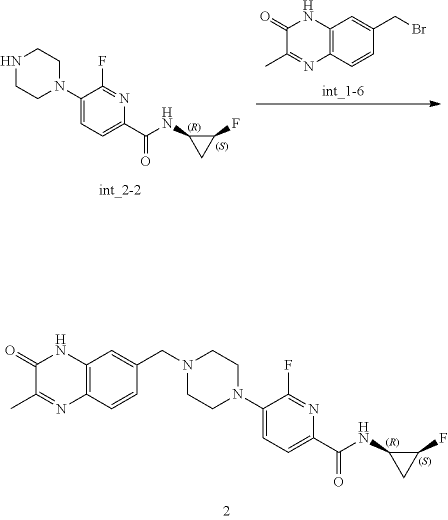

Compound int_2-2 (283 mg) was dissolved in acetonitrile (10 mL), and potassium carbonate (490 mg, 3.56 mmol) was added, followed by addition of int_1-6 (225 mg, 0.889 mmol). The reaction solution was purged with nitrogen, warmed to 60° C., and stirred for 2 h. After cooling, water (10 mL) was added to the reaction solution, and the mixture was extracted with dichloromethane/tetrahydrofuran (25/1, 50 mL×4). The organic phases were combined, dried over anhydrous sodium sulfate, and subjected to suction filtration. The filtrate was concentrated to dryness under reduced pressure, and the residue was purified by C18 preparative liquid chromatography to give compound 2 as a yellow solid.

LCMS m/z (ESI): 455.6 [M+H]⁺.

¹H NMR (400 MHz, DMSO-d₆) δ 12.27 (s, 1H), 8.38 (d, J=4.5 Hz, 1H), 7.87 (dd, J=8.1, 1.4 Hz, 1H), 7.65 (d, J=8.2 Hz, 1H), 7.58 (dd, J=10.6, 8.1 Hz, 1H), 7.28-7.21 (m, 2H), 4.86-4.64 (m, 1H), 3.61 (s, 2H), 3.19 (t, J=4.7 Hz, 4H), 2.85-2.78 (m, 1H), 2.56 (t, J=4.9 Hz, 4H), 2.39 (s, 3H), 1.32 (m, 1H), 1.13-1.02 (m, 1H).

### Example 3. Synthesis of Compound 3 and Compound 4

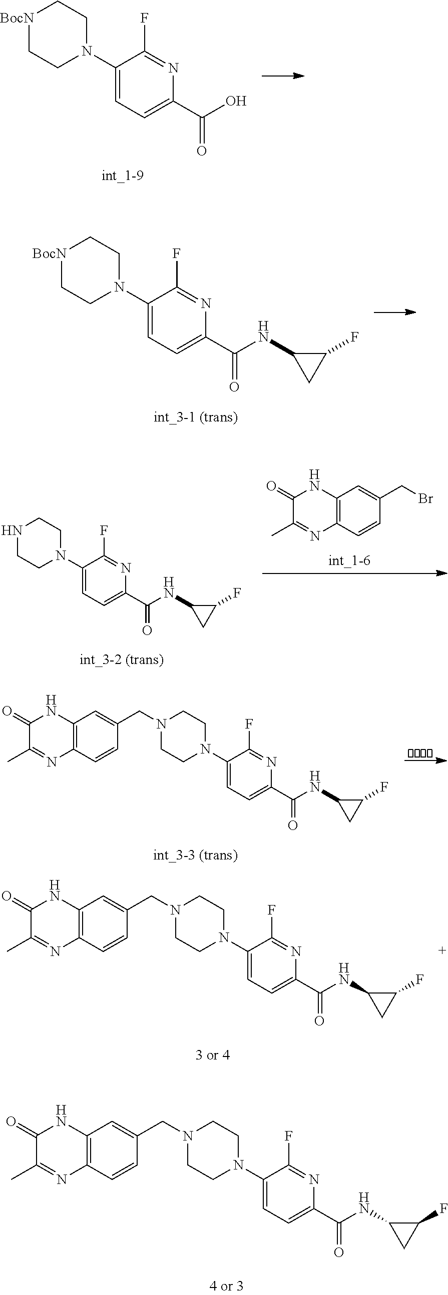

### Step 1: Synthesis of Compound Int_3-1:

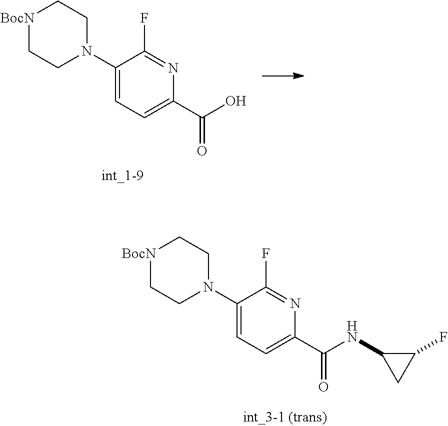

Intermediate int_1-9 (300 mg, 0.923 mmol) was dissolved in N,N-dimethylformamide (3 mL), and trans-2-fluorocyclopropanamine hydrochloride (123 mg, 1.108 mmol), N,N,N′,N′-tetramethyl-O-(7-azabenzotriazol-1-yl)uronium hexafluorophosphate (456.15 mg, 1.192 mmol), and N,N-diisopropylethylamine (476 mg, 3.69 mmol) were added. The reaction solution was stirred at 25° C. overnight. Water (20 mL) was added to the reaction solution, and the mixture was extracted with ethyl acetate (20 mL×3). The organic phases were combined, washed with saturated brine (30 mL×3), dried over anhydrous sodium sulfate, and filtered. The filtrate was concentrated under reduced pressure to give a crude product. The crude product was separated and purified by silica gel chromatography to give intermediate int_3-1 (trans mixture).

LCMS m/z (ESI): 383.2 [M+H]⁺.

### Step 2: Synthesis of Compound Int_3-2:

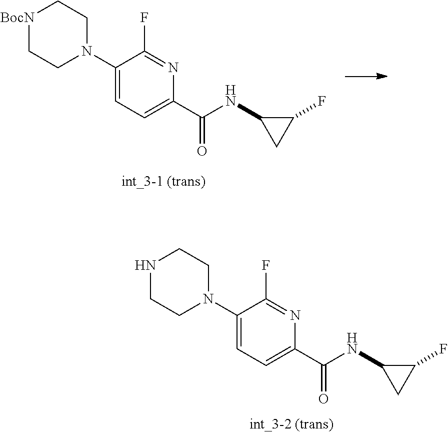

Compound int_3-1 (250 mg, 0.654 mmol) was dissolved in a solution of hydrogen chloride in dioxane (5 mL, 4 M), and the reaction solution was stirred at 25° C. for 2 h. The reaction solution was directly concentrated under reduced pressure to give crude compound int_3-2 as a light yellow solid (trans mixture).

LCMS m/z (ESI): 283.1 [M+H]⁺.

### Step 3: Synthesis of Compound Int_3-3:

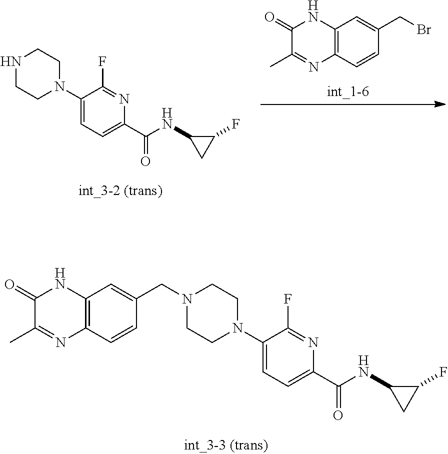

Compound int_3-2 (205 mg) was dissolved in acetonitrile (5 mL), and potassium carbonate (356 mg, 2.585 mmol) and compound int_1-6 (163 mg, 0.646 mmol) were added at room temperature. The reaction solution was purged thoroughly with nitrogen and stirred at 60° C. for 2 h. The reaction solution was cooled to room temperature and then poured into water, and the mixture was extracted with dichloromethane/tetrahydrofuran (10:1, 30 mL×6). The organic phases were combined, dried over anhydrous sodium sulfate, and concentrated under reduced pressure to give a crude product. The crude product was purified by C18 preparative liquid chromatography (formic acid system) to give product int_3-3 (formate, trans mixture) as a white solid.

LCMS m/z (ESI): 455.4 [M+H]⁺.

¹H NMR (400 MHz, DMSO-d₆) δ 12.27 (s, 1H), 8.52 (dd, J=4.9, 1.8 Hz, 1H), 8.15 (s, 1H), 7.85 (dd, J=8.1, 1.4 Hz, 1H), 7.65 (d, J=8.1 Hz, 1H), 7.57 (dd, J=10.6, 8.1 Hz, 1H), 7.28-7.21 (m, 2H), 4.83 (m, 1H), 3.61 (s, 2H), 3.26-3.22 (m, 1H), 3.19-3.15 (m, 4H), 2.59-2.53 (m, 4H), 2.39 (s, 3H), 1.33 (m, 1H), 1.13 (m, 1H).

### Step 4: Synthesis of Compound 3 and Compound 4:

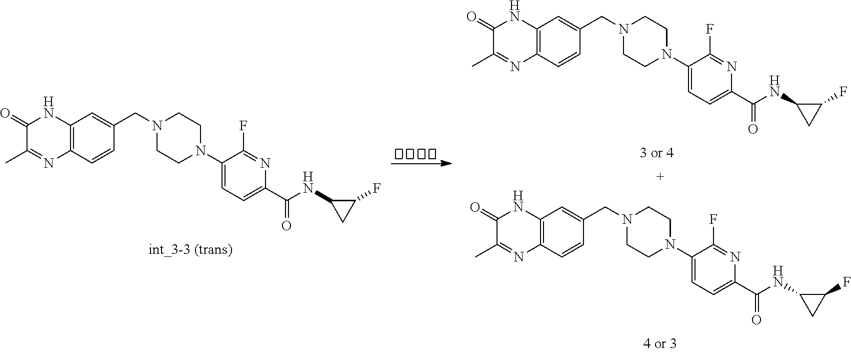

Compound int_3-3 was subjected to chiral resolution to give compound 3 (Rt=17.681 min) and compound 4 (Rt=19.998 min).

### Chiral Resolution Method:

Instrument: Waters Prep SFC 150
Chromatographic column: CHIRAL ART Cellulose-SC (3 cm×25 cm, 5 μm)
Mobile phase: CO₂:IPA (0.2% 2 mM NH₃-MeOH)=55:45
Elution gradient: isocratic
Flow rate: 80 mL/min
Column temperature: 35° C.
Detection wavelength: 220 nm

### Chiral Analysis Method:

Instrument: Agilent 126011
Chromatographic column: Chiralpak IC (4.6 mm I.D.×250 mmL, 5 Wm)
Mobile phase: (n-hexane:ethanol:diethylamine)=60:40:0.1
Elution gradient: isocratic
Flow rate: 1.0 mL/min
Column temperature: 30° C.
Detection wavelength: 220 nm

### Example 4. Synthesis of Compound 5

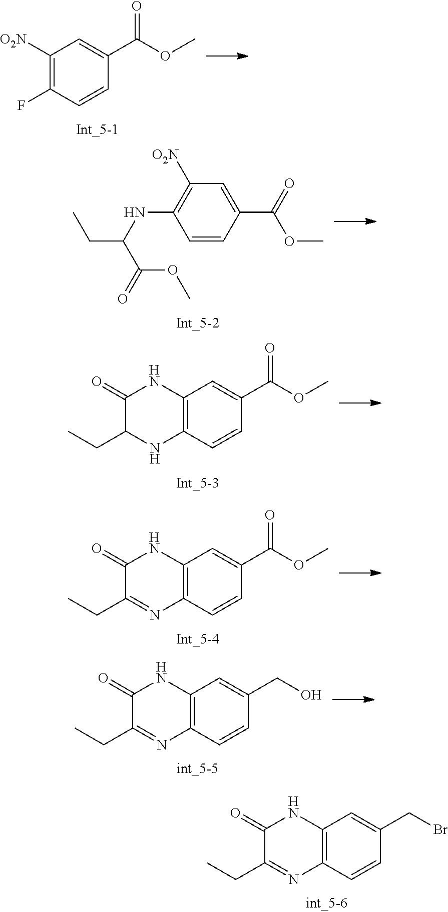

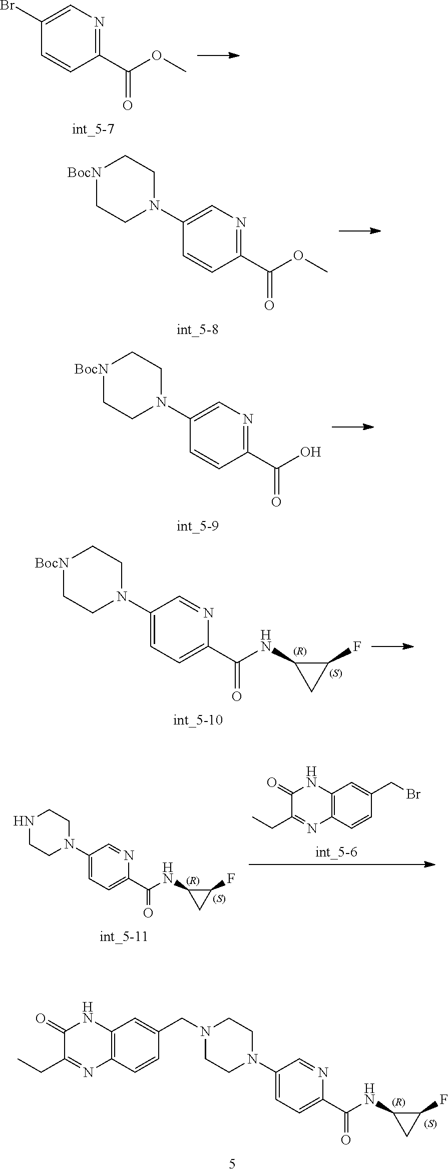

### Step 1: Synthesis of Compound Int_5-2:

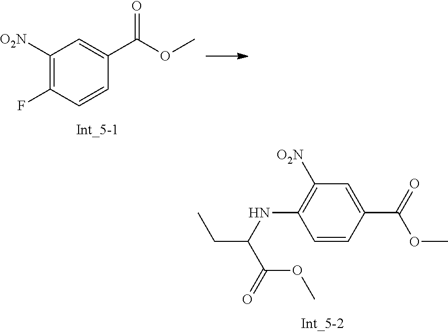 [SMILES: COC(=O)c1ccc(F)c([N+](=O)[O-])c1 ; CCC(Nc1ccc(C(=O)OC)cc1[N+](=O)[O-])C(=O)OC]

Compound methyl 4-fluoro-3-nitrobenzoate (25 g, 125.54 mmol) and DL-2-amino-N-butyric acid methyl ester hydrochloride (23.14 g, 150.65 mmol) were dissolved in tetrahydrofuran (150 mL), and sodium bicarbonate (42.19 g, 502.16 mmol) was added. The reaction solution was stirred at 25° C. for 16 h. Water (500 mL) was added to the reaction solution, and the mixed solution was extracted with ethyl acetate (500 mL×3). The organic phases were combined, dried over anhydrous magnesium sulfate, and subjected to suction filtration, and the filtrate was concentrated to dryness under reduced pressure to give crude compound int_5-2 as a yellow solid.

LCMS m/z (ESI): 297.0 [M+H]^{+.}

### Step 2: Synthesis of Compound Int_5-3:

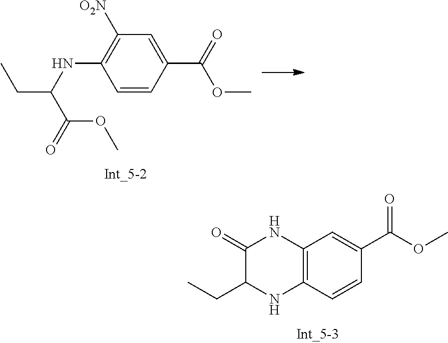 [SMILES: CCC(Nc1ccc(C(=O)OC)cc1[N+](=O)[O-])C(=O)OC ; CCC1Nc2ccc(C(=O)OC)cc2NC1=O]

Compound int_5-2 (11 g, 37.13 mmol) was dissolved in acetic acid (150 mL), and iron powder (20.73 g, 371.28 mmol) was added. The reaction solution was stirred at 50° C. for 2 h. After cooling, a saturated aqueous sodium carbonate solution (1.0 L) was added to the reaction solution, and the mixed solution was extracted with ethyl acetate (1.0 L×3). The organic phases were combined, dried over anhydrous magnesium sulfate, and subjected to suction filtration, and the filtrate was concentrated to dryness under reduced pressure to give compound int_5-3 as a white solid.

LCMS m/z (ESI): 235.0 [M+H]⁺.

### Step 3: Synthesis of Compound Int_5-4:

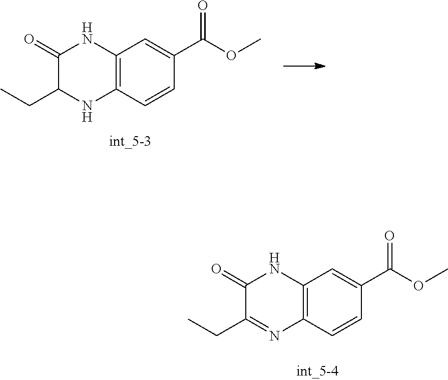 [SMILES: CCC1Nc2ccc(C(=O)OC)cc2NC1=O ; CCc1nc2ccc(C(=O)OC)cc2[nH]c1=O]

Compound int_5-3 (7.5 g, 32.02 mmol) was dissolved in dichloromethane (160 mL), and dichlorodicyanobenzoquinone (8.72 g, 38.42 mmol) was added. The reaction solution was stirred at 25° C. for 2 h. The reaction solution was concentrated to dryness under reduced pressure, slurried with methanol (35 mL) at 30° C. for 15 min, and filtered to give compound int_5-4 as a white solid.

LCMS m/z (ESI): 233.0 [M+H]⁺.

### Step 4: Synthesis of Compound Int_5-5:

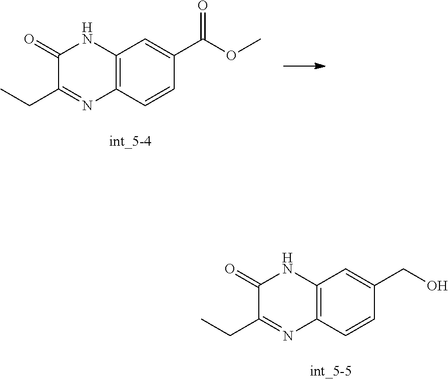

Compound int_5-4 (2 g, 8.61 mmol) was dissolved in tetrahydrofuran (90 mL), and lithium aluminum hydride (980 mg, 25.84 mmol) was added at 0° C. under nitrogen atmosphere. The reaction solution was stirred at 0° C. for 2 h. Sodium sulfate decahydrate was added to the reaction solution with stirring at 0° C. until no foam generation was observed, indicating complete quenching. The system was subjected to suction filtration, and the filter cake was rinsed with methanol/dichloromethane (1:10, 100 mL×3). The filtrate was concentrated under reduced pressure to give a crude product. The crude product was separated and purified by silica gel chromatography to give intermediate int_5-5.

MS m/z (ESI): 205.0 [M+H]⁺.

### Step 5: Synthesis of Compound Int_5-6:

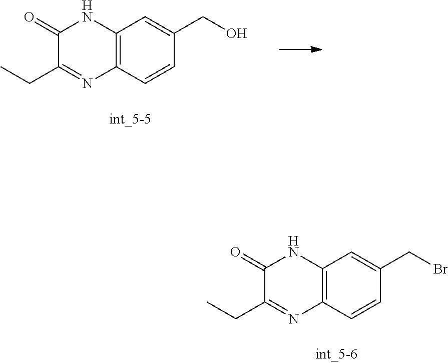

Compound int_5-5 (200 mg, 979.31 μmol) was dissolved in 48% hydrobromic acid (3 mL). The reaction solution was stirred at 80° C. for 16 h. After the reaction solution was cooled, water (30 mL) was added, and the mixture was directly filtered. The solid was collected, and product int_5-6 was obtained as a yellow solid.

LCMS m/z (ESI): 266.9 [M+H]⁺.

### Step 6: Synthesis of Compound Int_5-8:

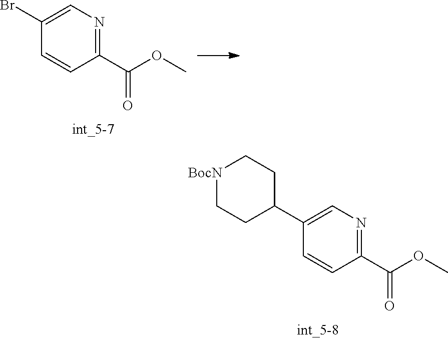 [SMILES: COC(=O)c1ccc(Br)cn1 ; COC(=O)c1ccc(C2CCN(C(=O)OC(C)(C)C)CC2)cn1]

Methyl 5-bromopyridine-2-carboxylate (1.00 g, 4.63 mmol) was dissolved in anhydrous methylbenzene (20 mL), and tert-butyl piperazine-1-carboxylate (1.03 g, 5.55 mmol) and anhydrous cesium carbonate (4.52 g, 13.89 mmol) were added. After the mixture was purged with nitrogen, palladium(II) acetate (77 mg, 0.46 mmol) and 1,1′-binaphthyl-2,2′-diphemyl phosphine (576 mg, 0.93 mmol) were added. The mixture was purged thoroughly with nitrogen again and stirred at 80° C. for 16 h under nitrogen atmosphere. The reaction solution was cooled and concentrated under reduced pressure to remove the solvent to give a crude product. The crude product was separated and purified by silica gel chromatography to give intermediate int_5-8.

LCMS m/z (ESI): 322.1 [M+H]⁺.

### Step 7: Synthesis of Compound Int_5-9:

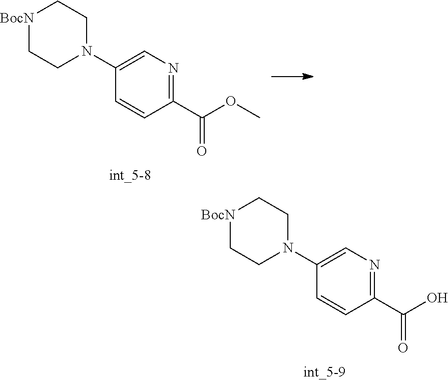 [SMILES: COC(=O)c1ccc(N2CCN(C(=O)OC(C)(C)C)CC2)cn1 ; CC(C)(C)OC(=O)N1CCN(c2ccc(C(=O)O)nc2)CC1]

Compound int_5-8 (300 mg, 0.93 mmol) was dissolved in methanol (3 mL), and a solution of sodium hydroxide (187 mg, 4.67 mmol) in water (2 mL) was added. The reaction solution was stirred at 20° C. for 2 h. The reaction solution was concentrated under reduced pressure to remove methanol, and water (20 mL) was added. The mixture was adjusted to pH 3-4 with 2 M diluted hydrochloric acid and extracted with ethyl acetate (20 mL×3). The organic phases were combined, washed with saturated brine (30 mL×2), dried over anhydrous sodium sulfate, and filtered. The filtrate was concentrated to give compound int_5-9 as a white solid.

LCMS m/z (ESI): 308.1 [M+H]⁺.

### Step 8: Synthesis of Compound Int_5-10:

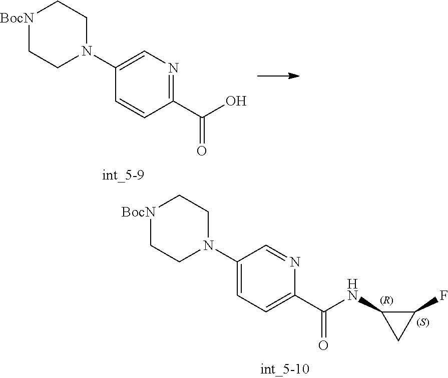

Compound int_5-9 (500 mg, 1.63 mmol) was dissolved in N,N-dimethylformamide (8 mL), and (1R,2S)-2-fluorocyclopropanamine 4-methylbenzenesulfonate (147 mg, 1.95 mmol), diisopropylethylamine (841 mg, 6.51 mmol), and O-(7-azabenzotriazol-1-yl)-N,N,N′,N′-tetramethyluronium hexafluorophosphate (742 mg, 1.95 mmol) were added. The reaction solution was stirred at 25° C. for 12 h. Water (100 mL) was added to the reaction solution, and the mixture was extracted with ethyl acetate (30 mL×3). The organic phases were combined, washed with saturated brine (50 mL×3), dried over anhydrous sodium sulfate, and filtered. The filtrate was concentrated to give a crude product. The crude product was separated and purified by silica gel chromatography to give intermediate int_5-10.

LCMS m/z (ESI): 365.2 [M+H]⁺.

### Step 9: Synthesis of Compound Int_5-11:

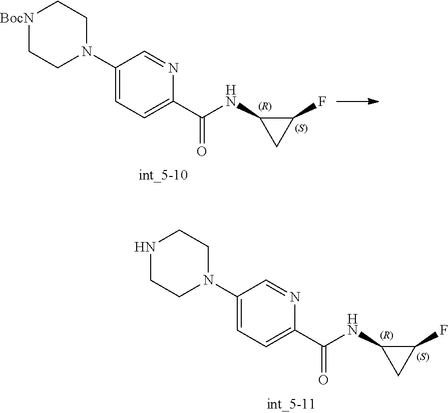

Compound int_5-10 (200 mg, 0.549 mmol) was dissolved in 1,4-dioxane (2 mL), and a solution of hydrogen chloride in dioxane (4 mL, 4 M) was slowly added dropwise. The reaction solution was stirred at 25° C. for 1 h. The reaction solution was directly concentrated to dryness under reduced pressure to give crude compound int_5-11 as a white solid.

LCMS m/z (ESI): 265.1 [M+H]⁺.

### Step 10: Synthesis of Compound 5:

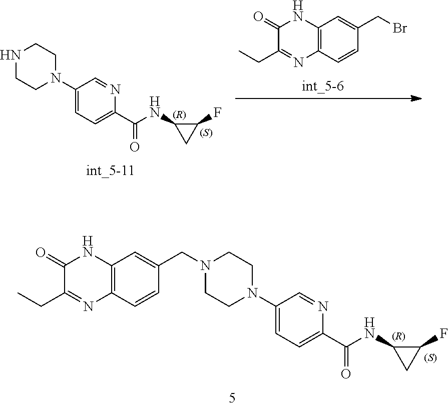

Compound int_5-11 (140 mg) was dissolved in acetonitrile (5 mL), and compound int_5-6 (124 mg, 0.465 mmol) and cesium carbonate (606 mg, 1.86 mmol) were added. The reaction solution was stirred at 60° C. for 1 h. After cooling, water (50 mL) was added to the reaction solution, and the mixture was extracted with dichloromethane (containing 10% tetrahydrofuran) (20 mL×3). The organic phases were combined, dried over anhydrous sodium sulfate, and filtered. The filtrate was concentrated to dryness under reduced pressure, and the crude product was purified by C18 preparative liquid chromatography (formic acid system) to give compound 5 (formate) as a white solid.

LCMS m/z (ESI): 451.7 [M+H]⁺.

¹H NMR (400 MHz, DMSO-d₆) δ 12.27 (s, 1H), 8.34 (d, J=4.8 Hz, 1H), 8.27 (d, J=2.9 Hz, 1H), 8.15 (s, 1H), 7.85 (d, J=8.7 Hz, 1H), 7.68 (d, J=8.1 Hz, 1H), 7.40 (dd, J=8.9, 2.9 Hz, 1H), 7.29-7.21 (m, 2H), 4.69-4.90 (m, 1H), 3.61 (s, 2H), 3.32-3.37 (m, 4H), 2.80-2.97 (m, 1H), 2.71-2.83 (m, 2H), 2.54 (m, 4H), 1.24 (m, 1H), 1.21 (t, J=7.4 Hz, 3H), 1.09-1.32 (m, 1H).

### Example 5. Synthesis of Compound 6

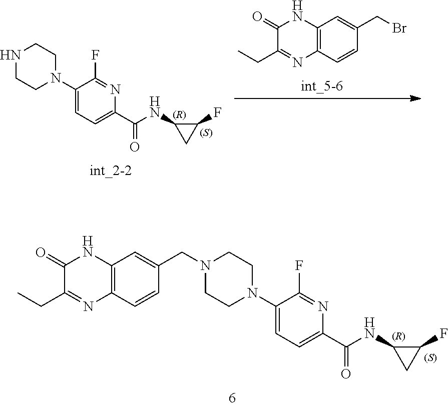

Compound int_2-2 (267 mg) was dissolved in acetonitrile (10 mL), and potassium carbonate (463 mg, 3.35 mmol) was added, followed by addition of compound int_5-6 (224 mg, 0.839 mmol). The reaction solution was purged with nitrogen, warmed to 60° C., and stirred for 2 h. After cooling, water (10 mL) was added to the reaction solution, and the mixture was extracted with dichloromethane/tetrahydrofuran (25/1, 50 mL×4). The organic phases were combined, dried over anhydrous sodium sulfate, and subjected to suction filtration. The filtrate was concentrated to dryness under reduced pressure, and then the residue was purified by C18 preparative liquid chromatography (formic acid system) to give compound 6 (formate) as a yellow solid.

LCMS m/z (ESI): 469.4 [M+H]⁺.

¹H NMR (400 MHz, DMSO-d₆) δ 12.27 (s, 1H), 8.38 (d, J=4.4 Hz, 1H), 8.15 (s, 1H), 7.86 (dd, J=8.0, 1.4 Hz, 1H), 7.68 (d, J=8.2 Hz, 1H), 7.58 (dd, J=10.6, 8.1 Hz, 1H), 7.28-7.22 (m, 2H), 4.85-4.65 (m, 1H), 3.61 (s, 2H), 3.18 (m, 4H), 2.84-2.76 (m, 3H), 2.56 (t, J=4.8 Hz, 4H), 1.32 (m, 1H), 1.21 (t, J=7.4 Hz, 3H), 1.07 (m, 1H).

### Example 6. Synthesis of Compound 7

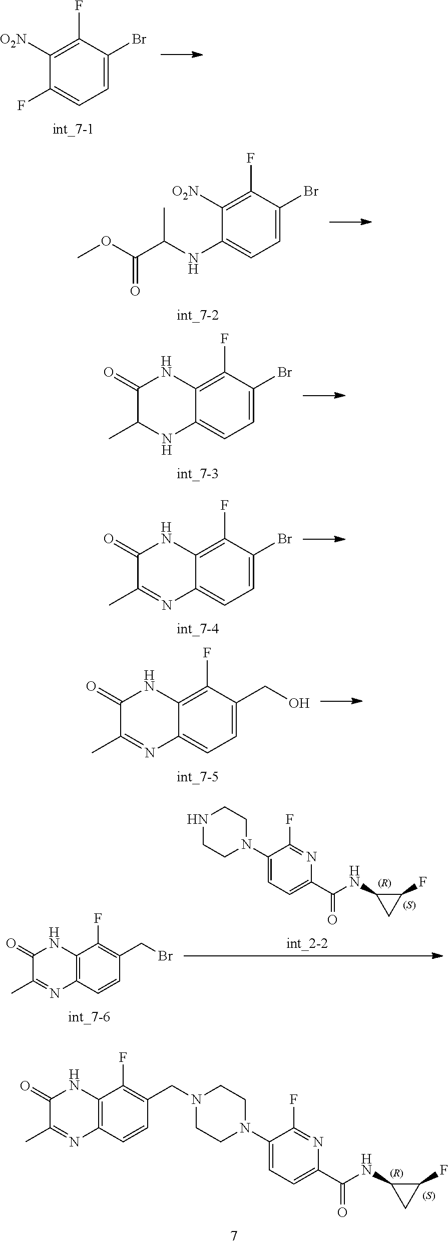

### Step 1: Synthesis of Compound Int_7-2:

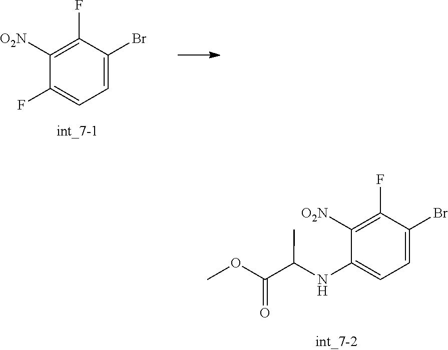 [SMILES: O=[N+]([O-])c1c(F)ccc(Br)c1F ; COC(=O)C(C)Nc1ccc(Br)c(F)c1[N+](=O)[O-]]

1-Bromo-2,4-difluoro-3-nitrobenzene (30 g, 126.06 mmol) and methyl 2-aminopropanoate (13.00 g, 126.06 mmol) were dissolved in N,N-dimethylformamide (400 mL), and then N,N-diisopropylethylamine (48.88 g, 378.17 mmol) was added to the reaction solution. The reaction solution was stirred at 25° C. for 18 h. The solvent was concentrated to dryness under reduced pressure, and water (300 mL) was added. The mixture was extracted with ethyl acetate (100 mL×3). The organic phases were combined, dried over anhydrous sodium sulfate, and subjected to suction filtration, and the filtrate was concentrated to dryness under reduced pressure to give compound int_7-2 as an orange solid.

LCMS m/z (ESI): 320.9 [M+H]⁺.

### Step 2: Synthesis of Compound Int_7-3:

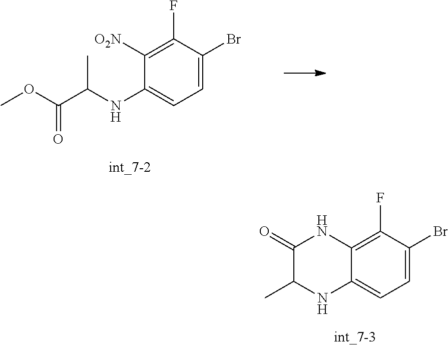 [SMILES: COC(=O)C(C)Nc1ccc(Br)c(F)c1[N+](=O)[O-] ; CC1Nc2ccc(Br)c(F)c2NC1=O]

Compound int_7-2 (12 g, 37.37 mmol) was dissolved in methanol (250 mL) and water (50 mL), and ammonium chloride (39.98 g, 747.43 mmol) was added. After the mixture was stirred at 25° C. for 2 min, iron powder (41.74 g, 747.43 mmol) was added, and the reaction system was purged 3 times with nitrogen. The reaction solution was stirred at 60° C. for 2 h. The reaction solution was filtered, concentrated under reduced pressure, diluted with water (100 mL), and extracted with ethyl acetate (100 mL×3). The organic phases were combined, dried over anhydrous sodium sulfate, and subjected to suction filtration, and the filtrate was concentrated to dryness under reduced pressure and slurried with ethyl acetate (50 mL) to give compound int_7-3 as a yellow solid.

LCMS m/z (ESI): 258.8 [M+H]⁺.

### Step 3: Synthesis of Compound Int_7-4:

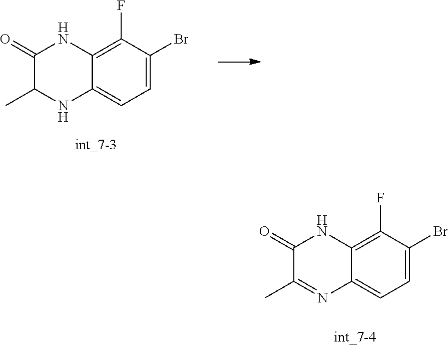

Compound int_7-3 (7.7 g, 29.72 mmol) and dichlorodicyanobenzoquinone (8.10 g, 35.67 mmol) were dissolved in anhydrous dichloromethane (500 mL). The reaction solution was stirred at 25° C. for 2 h. The reaction solution was concentrated to dryness under reduced pressure, and a saturated aqueous sodium bicarbonate solution (300 mL) was added. The mixture was stirred at 25° C. for 16 h and filtered to give a solid crude product, which was purified by slurrying with methyl tert-butyl ether (50 mL) to give compound int_7-4 as a gray solid.

LCMS m/z (ESI): 258.7 [M+H]⁺.

### Step 4: Synthesis of Compound Int_7-5:

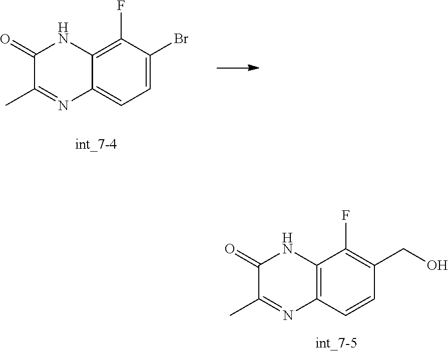

Compound int_7-4 (750 mg, 2.92 mmol) and 1-(tributylstannyl)methanol (1.03 g, 3.21 mmol) were dissolved in dioxane (20 mL), and chloro(2-dicyclohexylphosphino-2′,4′,6′-triisoporpyl-1,1′-biphenyl)[2-(2′-amino-1,1′-biphenyl)]palladium(II) (115 mg, 145.88 μmol) was added to the reaction solution under nitrogen atmosphere. The reaction solution was stirred at 80° C. for 16 h. The reaction solution was cooled to room temperature, and potassium fluoride (2 M, 20 mL) and hydrochloric acid (2 M, 10 mL) were added. The mixture was stirred at 25° C. for 30 min. The mixture was extracted with ethyl acetate (10 mL×3). The organic phases were combined, dried over anhydrous sodium sulfate, and subjected to suction filtration, and the filtrate was concentrated to dryness under reduced pressure. The crude product was separated and purified by silica gel chromatography to give intermediate int_7-5.

LCMS m/z (ESI): 208.8 [M+H]⁺.

### Step 5: Synthesis of Compound Int_7-6:

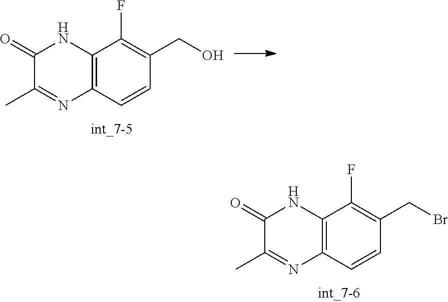

Compound int_7-5 (400 mg, 1.92 mmol) was dissolved in dichloromethane (40 mL), and carbon tetrabromide (2.10 g, 6.34 mmol) and trimethylphosphine (1 M, 11.53 mL) were added under nitrogen atmosphere in an ice bath. The reaction solution was stirred at 25° C. for 3 h under nitrogen atmosphere. The reaction solution was concentrated to dryness under reduced pressure, and the crude product was purified by slurrying with methyl tert-butyl ether (5 mL) to give compound int_7-6 as a green solid.

¹H NMR (400 MHz, DMSO-d₆) δ 12.47 (s, 1H), 7.58-7.43 (m, 1H), 7.42-7.30 (m, 1H), 4.88 (s, 2H), 2.42 (s, 3H).

### Step 6: Synthesis of Compound 7:

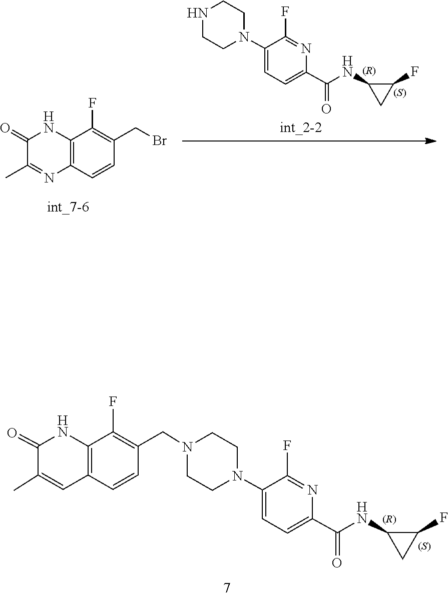

Compound int_2-2 (76 mg) was dissolved in N,N-dimethylformamide (3 mL), and potassium carbonate (187 mg, 1.345 mmol) and compound int_7-6 (73 mg, 0.269 mmol) were then added. The reaction solution was warmed to 60° C. and stirred for 5 h. The reaction solution was concentrated to dryness under reduced pressure and then purified by C18 preparative liquid chromatography to give compound 7 as a yellow solid.

LCMS m/z (ESI): 473.3 [M+H]⁺.

¹H NMR (400 MHz, DMSO-d₆) δ 12.42 (s, 1H), 8.35 (d, J=4.5 Hz, 1H), 7.84 (dd, J=8.0, 1.4 Hz, 1H), 7.59-7.42 (m, 2H), 7.26 (t, J=7.6 Hz, 1H), 4.86-4.61 (m, 1H), 3.68 (s, 2H), 3.15 (t, J=4.9 Hz, 4H), 2.86-2.74 (m, 1H), 2.57 (s, 4H), 2.39 (s, 3H), 1.35-1.28 (m, 1H), 1.12-0.97 (m, 1H).

### Example 7. Synthesis of Compound 8

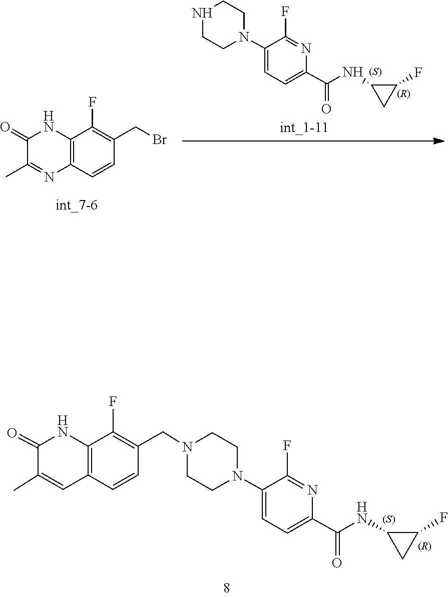

Compound int_1-11 (80 mg) was dissolved in N,N-dimethylformamide (3 mL), and potassium carbonate (187 mg, 1.345 mmol) and compound int_7-6 (85 mg, 0.314 mmol) were then added. The reaction solution was warmed to 60° C. and stirred for 4 h. The reaction solution was concentrated to dryness under reduced pressure and then purified by C18 preparative liquid chromatography to give compound 8 as a yellow solid.

LCMS m/z (ESI): 472.9 [M+H]⁺.

¹H NMR (400 MHz, DMSO-d₆) δ 12.68-12.20 (brs, 1H), 8.35 (d, J=4.4 Hz, 1H), 7.86 (dd, J=8.1, 1.4 Hz, 1H), 7.61-7.49 (m, 2H), 7.29 (dd, J=8.4, 7.0 Hz, 1H), 4.86-4.61 (m, 1H), 3.70 (s, 2H), 3.18 (t, J=4.9 Hz, 4H), 2.86-2.79 (m, 1H), 2.59 (t, J=5.0 Hz, 4H), 2.41 (s, 3H), 1.38-1.24 (m, 1H), 1.13-1.01 (in, 1H).

Target compounds 9-66 in Table 1 were obtained by using the synthesis methods in Example 1-7 or other similar routes with different starting materials.

TABEL 1

| Com- | | MS | Com- | | MS |
|---|---|---|---|---|---|
| pound | Compound structure | (M + H)⁺ | pound | Compound structure | (M + H)⁺ |
| 9 | 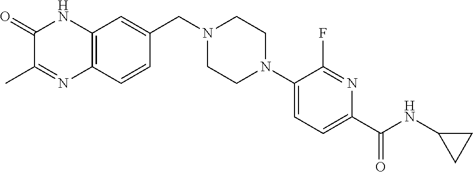 [SMILES: Cc1nc2ccc(CN3CCN(c4ccc(C(=O)NC5CC5)nc4F)CC3)cc2[nH]c1=O] | 437 | 10 | 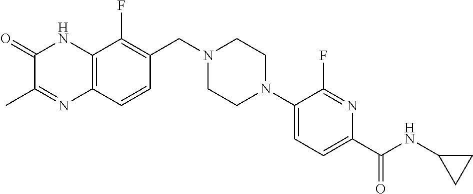 [SMILES: Cc1nc2ccc(CN3CCN(c4ccc(C(=O)NC5CC5)nc4F)CC3)c(F)c2[nH]c1=O] | 455 |
| 11 | 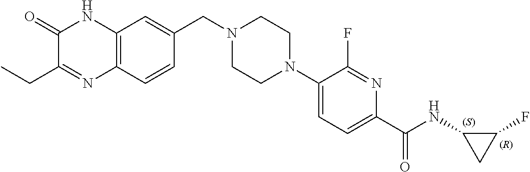 | 469 | 12 | 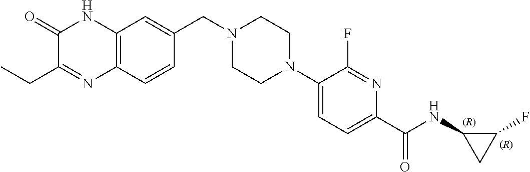 | 469 |
| 13 | 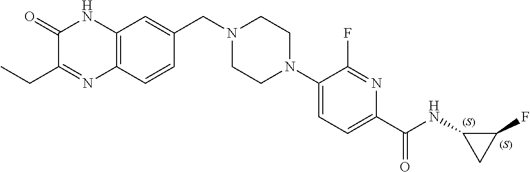 | 469 | 14 | 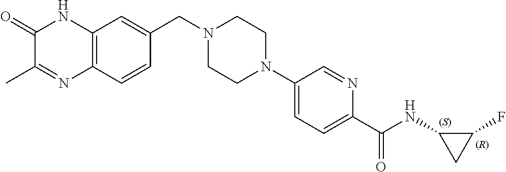 | 437 |
| 15 | 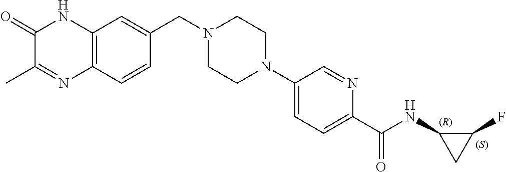 | 437 | 16 | 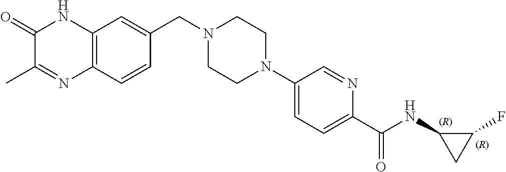 | 437 |
| 17 | 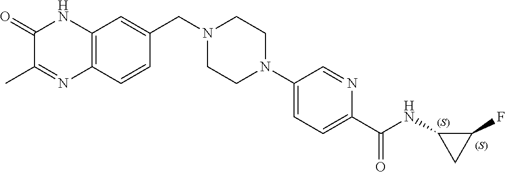 | 437 | 18 | 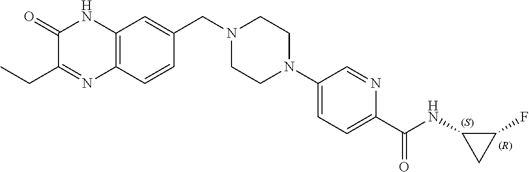 | 451 |
| 19 | 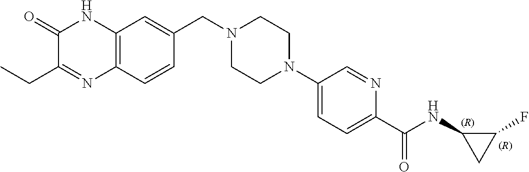 | 451 | 20 | 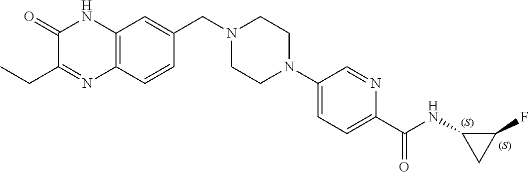 | 451 |
| 21 | 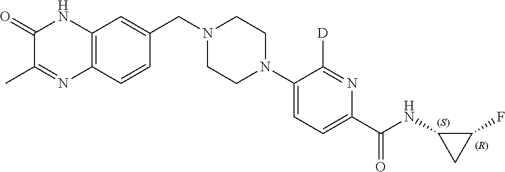 | 438 | 22 | 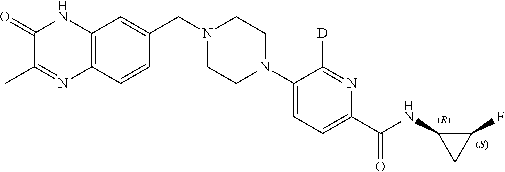 | 438 |
| 23 | 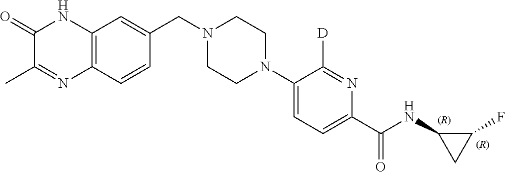 | 438 | 24 | 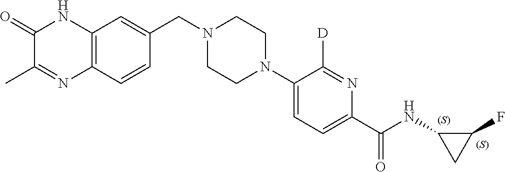 | 438 |
| 25 | 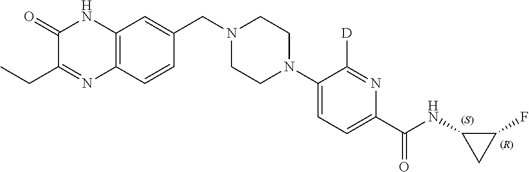 | 452 | 26 | 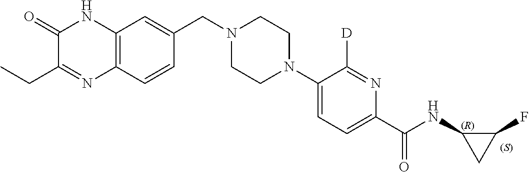 | 452 |
| 27 | 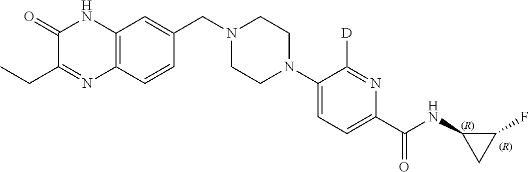 | 452 | 28 | 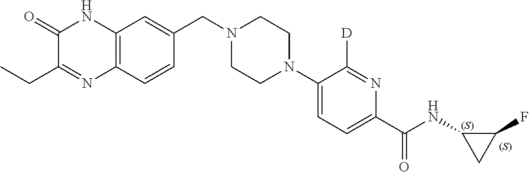 | 452 |
| 29 | 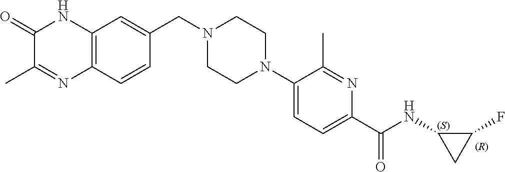 | 451 | 30 | 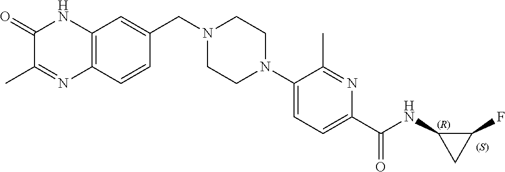 | 451 |
| 31 | 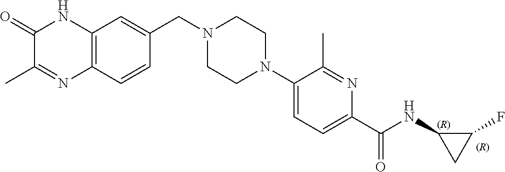 | 451 | 32 | 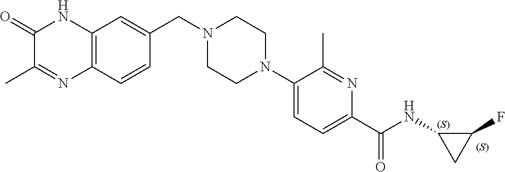 | 451 |
| 33 | 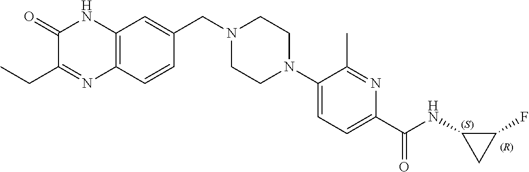 | 465 | 34 | 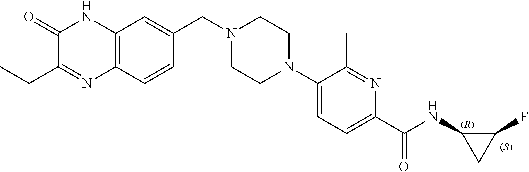 | 465 |
| 35 | 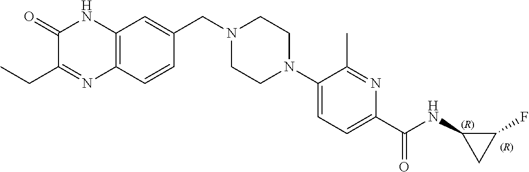 | 465 | 36 | 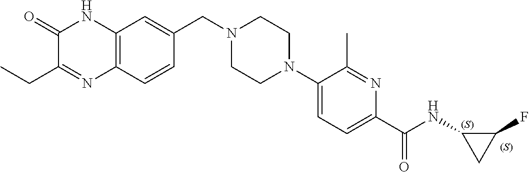 | 465 |
| 37 | 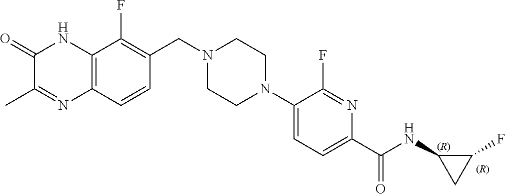 | 473 | 38 | 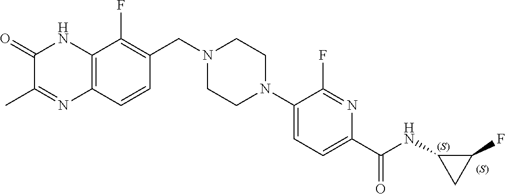 | 473 |
| 39 | 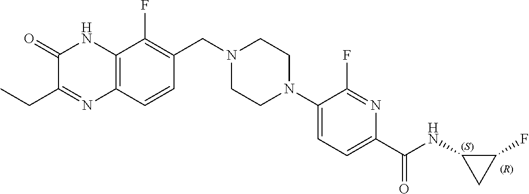 | 487 | 40 | 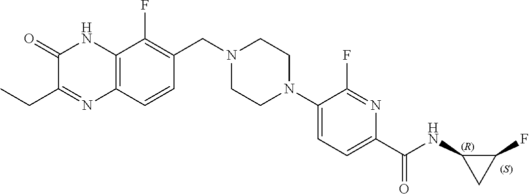 | 487 |
| 41 | 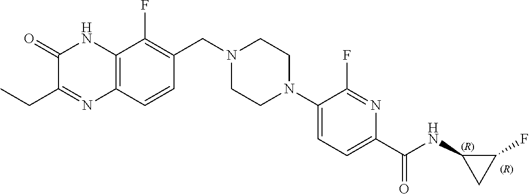 | 487 | 42 | 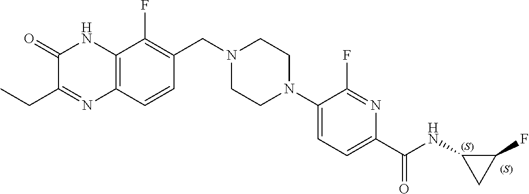 | 487 |
| 43 | 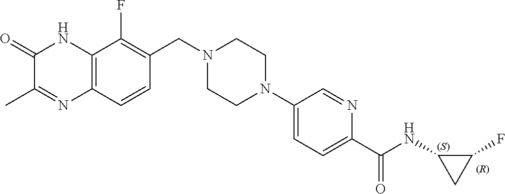 | 455 | 44 | 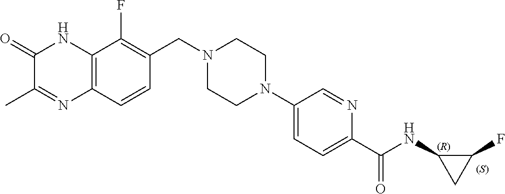 | 455 |
| 45 | 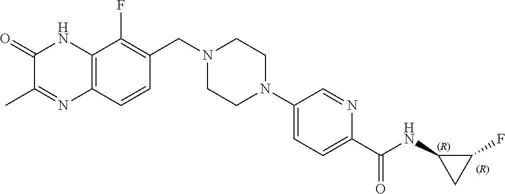 | 455 | 46 | 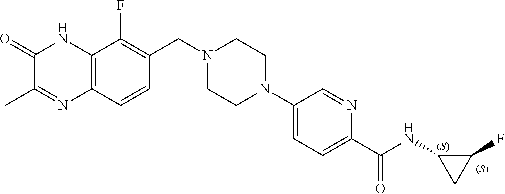 | 455 |
| 47 | 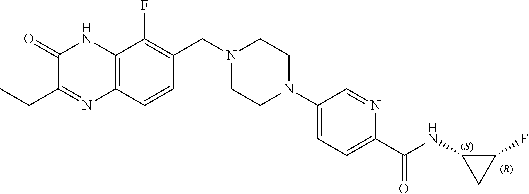 | 469 | 48 | 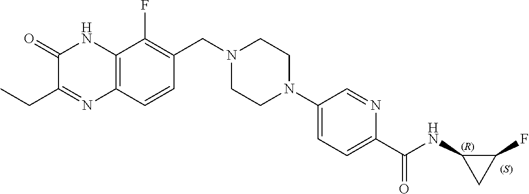 | 469 |
| 49 | 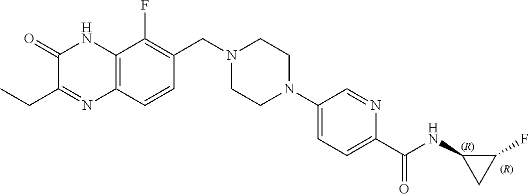 | 469 | 50 | 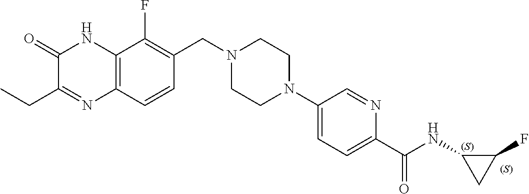 | 469 |
| 51 | 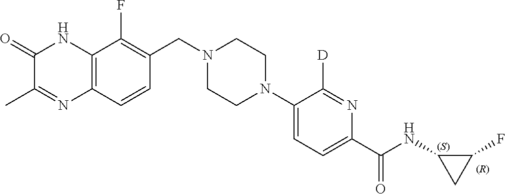 | 456 | 52 | 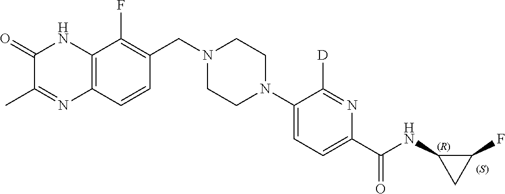 | 456 |
| 53 | 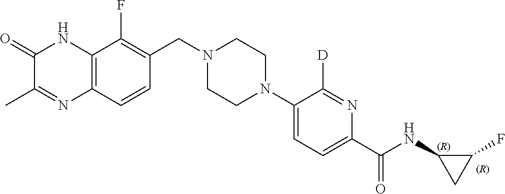 | 456 | 54 | 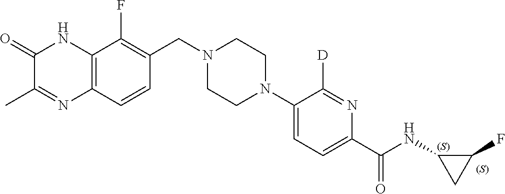 | 456 |
| 55 | 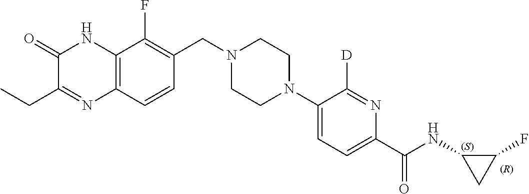 | 470 | 56 | 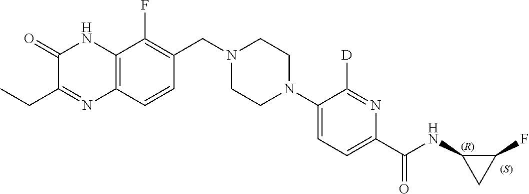 | 470 |
| 57 | 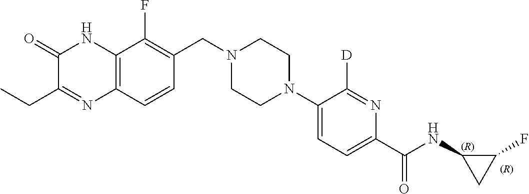 | 470 | 58 | 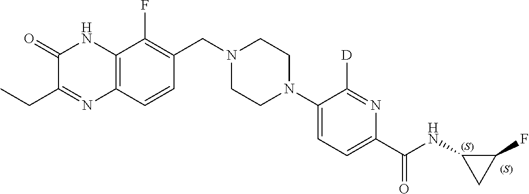 | 470 |
| 59 | 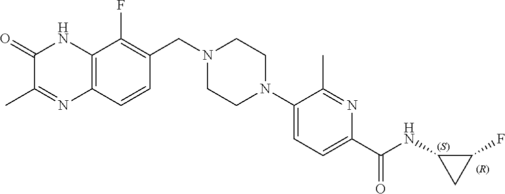 | 469 | 60 | 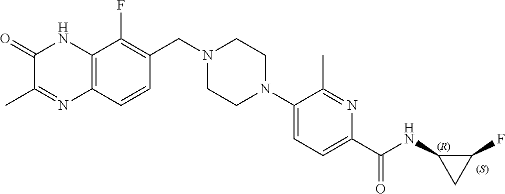 | 469 |
| 61 | 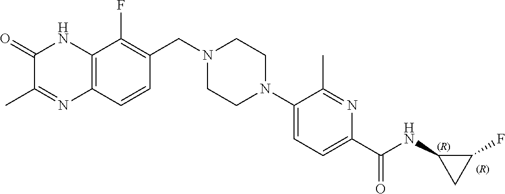 | 469 | 62 | 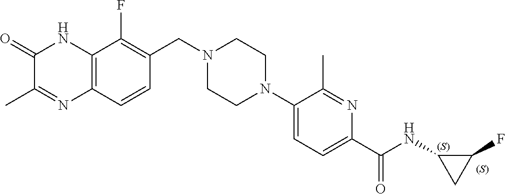 | 469 |
| 63 | 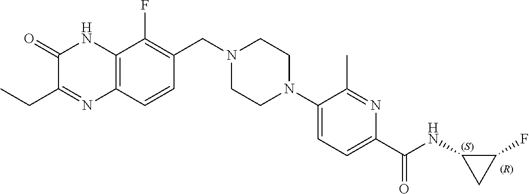 | 483 | 64 | 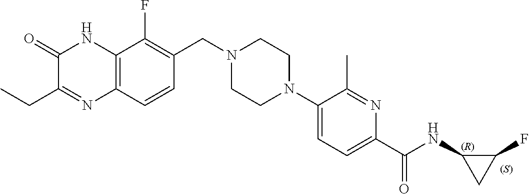 | 483 |
| 65 | 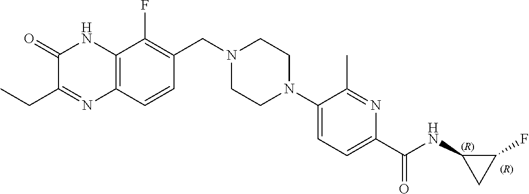 | 483 | 66 | 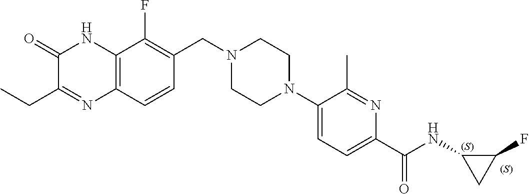 | 483 |

### Biological Example 1. Experiment on In Vitro Inhibitory Activity of Compounds of the Present Disclosure Against Poly(ADP-Ribose) Polymerase [PARP-1 and PARP-2 Enzymes]

A 384-well plate was coated with histones at 4° C. overnight, rinsed three times with a PBST buffer, and blocked at room temperature for 1 h. After 1 h, the plate was rinsed three more times with PBST. DMSO or a serially diluted compound, and a mixture containing the PARP-1 or PARP-2 enzyme and DNA were added. The plate was incubated at 25° C. for 10 min. After 10 min, NAD⁺ was added to start the reaction. After reacting at room temperature for 60 min, the plate was rinsed three times with PBST, and the level of poly/mono-ADP ribose on the histones was detected by the addition of a poly/mono-ADP ribose antibody conjugated with horseradish peroxidase (HRP). After incubation at room temperature for 1 h, HRP substrates ECL A and B were added, and Envision quantified chemiluminescence. The inhibition percentages and IC₅₀ of the compounds were calculated compared to the control group DMSO. The results are shown in Table 2 below.

TABLE 2
Inhibitory activity of the compounds of the
present disclosure against PARP (IC₅₀, nM)

| Compound | PARP-1 | PARP-2 | Compound | PARP-1 | PARP-2 |
|---|---|---|---|---|---|
| 1 | 1.1 | 524 | 2 | 1.2 | 935 |
| 5 | 1.1 | 112 | 6 | 1.5 | 203 |
| 9 | 1.2 | 944 | AZD9574 | 1.3 | >2000 |

### Biological Example 2. Experiment on In Vitro Inhibition of MDA-MB-436 Cell Proliferation by Compounds of the Present Disclosure

MDA-MB-436 cells were seeded in a 96-well low-adsorption plate at 1200 cells/well. After the cells adhered to the wall overnight, the serially diluted compound was added, and the cells were incubated for another 10 days. The ATP level of the cells was then determined by CTG to evaluate the cell growth. The growth inhibition percentages and IC₅₀ of the compounds were calculated compared to the DMSO group. The specific results are shown in Table 3 below.

TABLE 3
Inhibitory activity of the compounds of the present disclosure against MDA-MB-436 cells
(IC₅₀, nM)

| Compound | IC₅₀ | Compound | IC₅₀ | Compound | IC₅₀ | Compound | IC₅₀ |
|---|---|---|---|---|---|---|---|
| 1 | 1.6 | 2 | 2.4 | 3 | 2.0 | 4 | 1.9 |
| 6 | 2.6 | 7 | 10 | 8 | 7.6 | 9 | 3.7 |
| 10 | 16 | 11 | 1.7 | AZD9574 | 9 | | |

### Biological Example 3. Experiment on In Vitro Liver Microsomal Stability of Compounds of the Present Disclosure

After 1 μM compound was incubated with 500 μg/mL liver microsomes and a NADPH solution for varying time periods, the remaining amount of the compound was detected by LC-MS, and T_{1/2} and clearance rate were calculated. The specific results are shown in Table 4 below.

TABLE 4
Results of in vitro liver microsomal stability evaluation
of the compounds of the present disclosure (T_{1/2}, min)

| | | Mouse liver | | Human liver | Mouse liver |
|---|---|---|---|---|---|
| | Human liver | microsome | | microsome | microsome |
| Compound | microsome T_{1/2} | T_{1/2} | Compound | T_{1/2} | T_{1/2} |
| 1 | 494 | 293 | 2 | 233 | 309 |
| 7 | 127 | 90 | 8 | 95 | 129 |
| 9 | 228 | 566 | 10 | 100 | 863 |
| AZD9574 | 103 | 126 | | | |

### Biological Example 4. In Vivo Pharmacokinetic Experiment of Compounds of the Present Disclosure

CD-1 female mice aged 7 to 10 weeks were intravenously administered and orally administered at doses of 1 mg/kg and 10 mg/kg, respectively. The mice were fasted for at least 12 h before administration and given food 4 h after administration, and they were given free access to water throughout the experiment.

On the day of the experiment, animals in the intravenous group were given the corresponding compound by single injection via the tail vein at an administration volume of 2 mL/kg, and animals in the oral group were given the corresponding compound by single intragastric injection at an administration volume of 10 mL/kg. The animals were weighed before administration, and their administration volume was calculated according to their body weight. The sample collection time points were 0.083 h, 0.167 h, 0.5 h, 1 h, 2 h, 4 h, 8 h, and 24 h. About 200 L of whole blood was collected through the orbital venous plexus at each time point and used to prepare plasma for concentration determination by high performance liquid chromatography-tandem mass spectrometry (LC-MS/MS). All animals were suffocated by CO₂ after the PK samples were collected at the last time point. The plasma concentrations were processed using a non-compartmental model of Winnolin™ version 8.2 (Pharsight, mountain View, CA) pharmacokinetic software, and the pharmacokinetic parameters were calculated using a linear-log trapezoidal method. The specific results are shown in Table 5 below.

TABLE 5
Results of in vivo pharmacokinetic evaluation of compounds of the present disclosure

| Route of | Pharmacokinetic | Compound | | Compound | Compound | Compound | Compound | |
|---|---|---|---|---|---|---|---|---|
| administration | parameter | 1 | Compound2 | 8 | 7 | 9 | 10 | AZD9574 |
| Injection (1 | V_{dss} (L/Kg) | 1.13 | 0.76 | 3.54 | 2.12 | 0.73 | 2.38 | 2.03 |
| mg/kg) | T_{1/2} (h) | 3.13 | 1.48 | 4.76 | 3.82 | 3.41 | 5.07 | 4.54 |
| | Cl (mL/min/kg) | 4.52 | 7.70 | 14 | 10.5 | 4.62 | 7.5 | 6.42 |
| | AUC₀₋ₗₐₛₜ | 3675 | 2121 | 1170 | 1572 | 3593 | 2172 | 2550 |
| | (h * ng/mL) | | | | | | | |
| Oral | Cₘₐₓ (ng/ml) | 14767 | 10620 | 3043 | 5593 | 16017 | 4533 | 5140 |
| administration | Tₘₐₓ (h) | 0.17 | 0.5 | 0.17 | 0.17 | 0.08 | 0.17 | 0.5 |
| (10 mg/kg) | T_{1/2} (h) | 2.08 | 1.71 | 3.16 | 2.53 | 1.89 | 3.8 | 4.37 |
| | AUC₀₋ₗₐₛₜ | 60800 | 30889 | 11256 | 16271 | 60783 | 20389 | 25433 |
| | (h * ng/ml) | | | | | | | |
| | F % | 166 | 146 | 95 | 103 | 192 | 93 | 99.5 |

### Biological Example 5. Experiment on In Vivo Efficacy of Some of Compounds of the Present Disclosure in MDA-MB-436 Xenograft Tumor Model

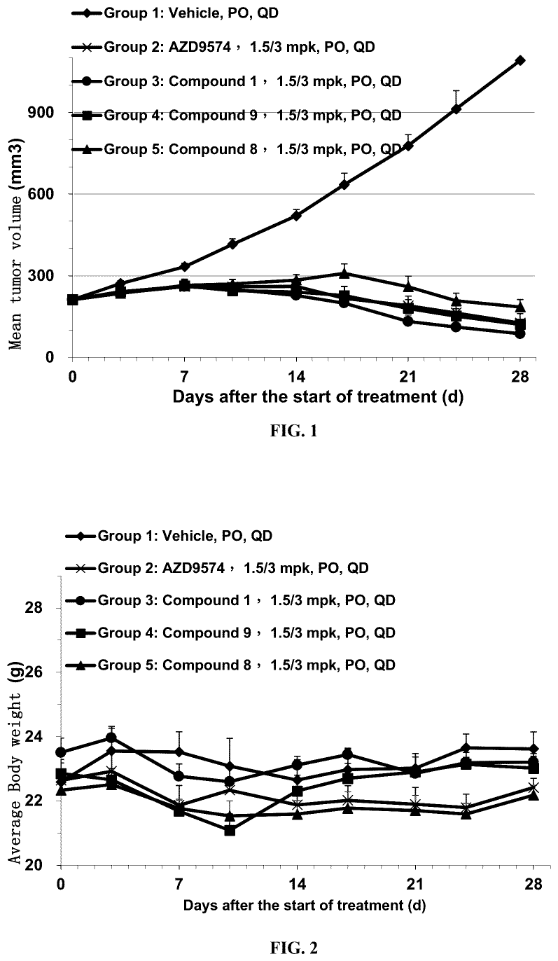

MDA-MB-436 cells were subjected to adherent culture in an L-15 medium containing 10% fetal bovine serum and 1% AA at 37° C. with 5% CO2. The cells were digested with trypsin and routinely passaged twice a week. When cells were maintained in the exponential growth phase with viability greater than 95%, the cells were collected, counted, and inoculated. Each 6- to 8-week-old female BALB/c nude mouse was inoculated with 10×106 MDA-MB-436 cells+Matrigel in a volume of 0.2 mL in the right cervicodorsal region. When the mean tumor volume reached 200-250 mm3, the mice were randomly divided into four groups (control group, AZD9574, compound 1, compound 8, and compound 9), with 5 mice in each group. The compounds were orally administered once a day, and the tumor volume and body weight of the mice were measured twice a week. After 28 days of administration, the mice were sacrificed. Data were processed using software such as Excel. Calculation of tumor growth inhibition (TGI) rates (%) of the compounds: When no tumor regression occurred, TGI (%)=[(1−(mean tumor volume at the end of administration in a treatment group−mean tumor volume at the start of administration of the treatment group))/(mean tumor volume at the end of treatment of the solvent control group−mean tumor volume at the start of treatment of the solvent control group)]×100%. When tumor regression occurred, TGI (%)=[1−(mean tumor volume at the end of administration in a treatment group−mean tumor volume at the start of administration of the treatment group)/mean tumor volume at the start of administration of the treatment group]×100%. The specific results are shown in Table 6, FIG. 1, and FIG. 2.

TABLE 6
Tumor growth inhibition rates of the compounds of the present
disclosure in a MDA-MB-436 xenograft tumor model

| | | | | Tumor |
|---|---|---|---|---|
| | | | | growth |
| | Mean tumor volume (mm³) | | | inhibition |
| Grouping | 0 days | 7 days | 28 days | rate |
| Vehicle | 212 | 333 | 1091 | — |
| AZD9574 | 212 | 260 | 126 | 141% |
| Compound 1 | 212 | 260 | 87 | 159% |
| Compound 8 | 212 | 265 | 121 | 143% |
| Compound 9 | 212 | 262 | 185 | 113% |

(Note:
the dose was 1.5 mg/kg from day 1 to day 7 and 3 mg/kg from day 8 to day 28.)

Although specific embodiments of the present invention have been described above, it will be appreciated by those skilled in the art that these embodiments are merely illustrative and that many changes or modifications can be made to these embodiments without departing from the principles and spirit of the present invention. The protection scope of the present invention is therefore defined by the appended claims.

1. A compound represented by general formula (1) or an isomer thereof, a crystalline form thereof, a pharmaceutically acceptable salt thereof, a hydrate thereof, or a solvate thereof: 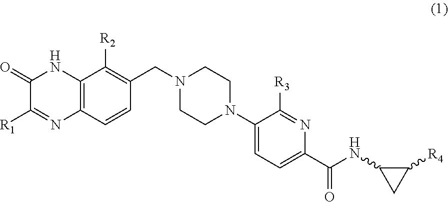
wherein in general formula (1):
R₁ is (C1-C3) alkyl, (C1-C3) fluoroalkyl, or (C3-C4) cycloalkyl;
R₂ is H or F;
R₃ is H, D, halogen, (C1-C3) alkyl, or (C1-C3) fluoroalkyl;
R₄ is H or F;
when R₄ is F, fluorocyclopropyl of 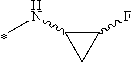
is a single isomer containing two chiral centers.

2. The compound or the isomer thereof, the crystalline form thereof, the pharmaceutically acceptable salt thereof, the hydrate thereof, or the solvate thereof according to claim 1, wherein in general formula (1),

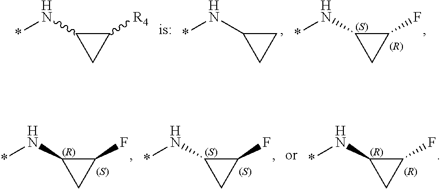

3. The compound or the isomer thereof, the crystalline form thereof, the pharmaceutically acceptable salt thereof, the hydrate thereof, or the solvate thereof according to claim 1, wherein in general formula (1), R₁ is methyl, ethyl, or cyclopropyl, preferably methyl or ethyl, and more preferably methyl.

4. The compound or the isomer thereof, the crystalline form thereof, the pharmaceutically acceptable salt thereof, the hydrate thereof, or the solvate thereof according to claim 1, wherein in general formula (1), R₃ is H, D, F, Cl, or methyl, preferably H, D, F, or methyl, more preferably H, D, or F, and more preferably F.

5. The compound or the isomer thereof, the crystalline form thereof, the pharmaceutically acceptable salt thereof, the hydrate thereof, or the solvate thereof according to claim 1, wherein the compound has one of the following structures:

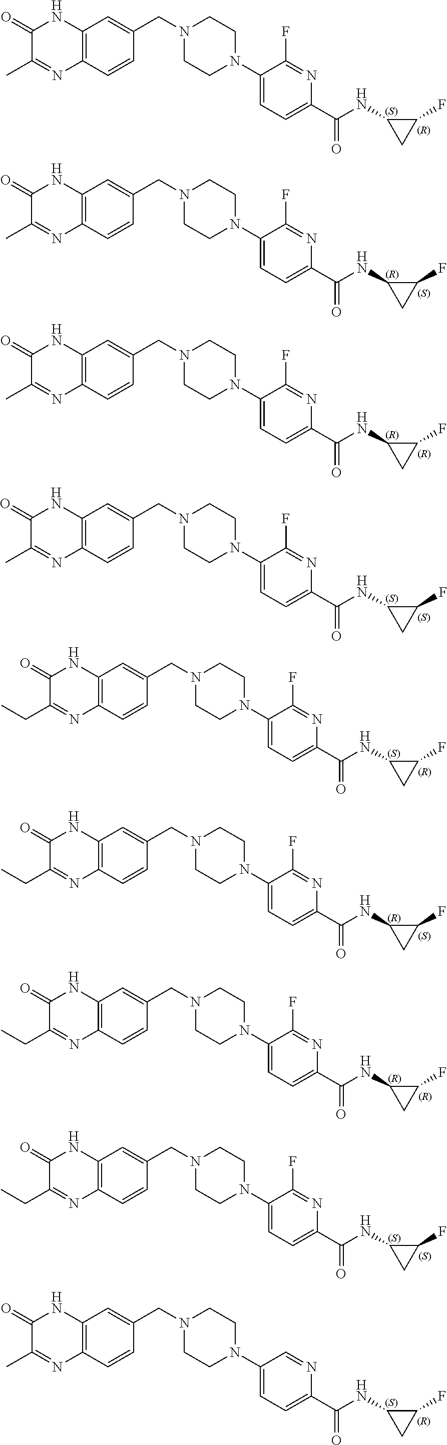

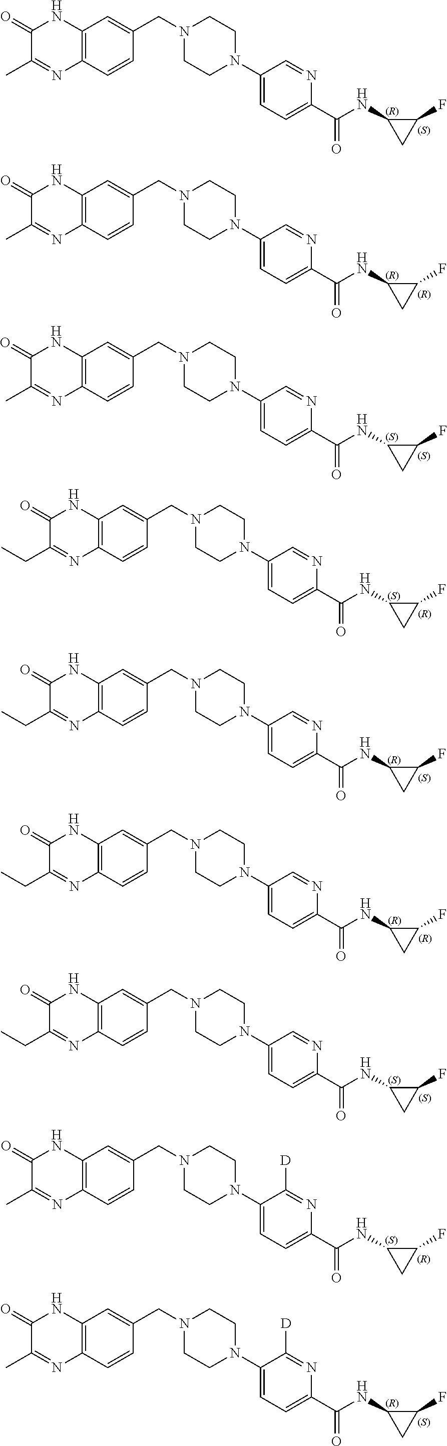

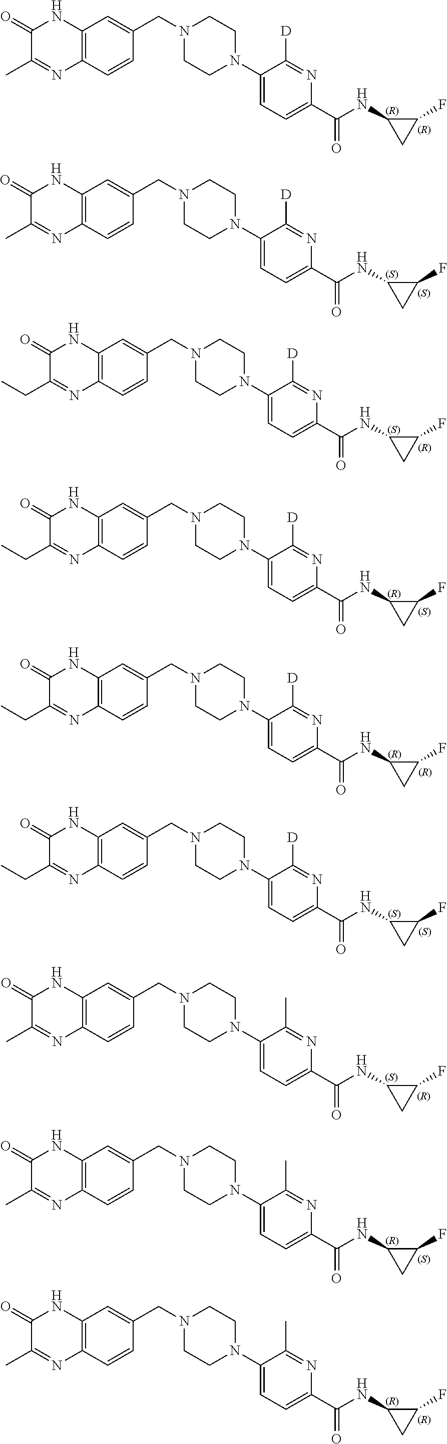

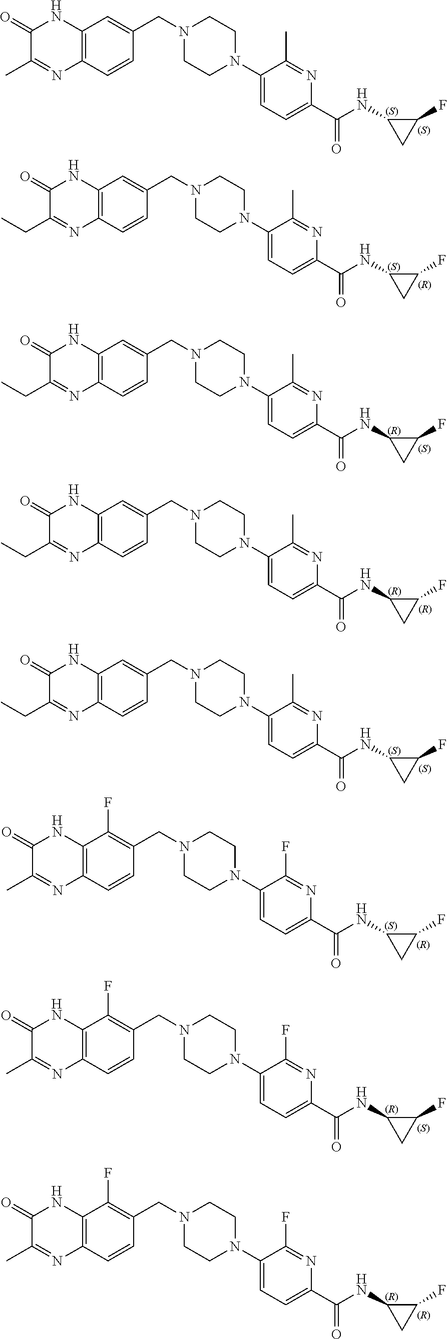

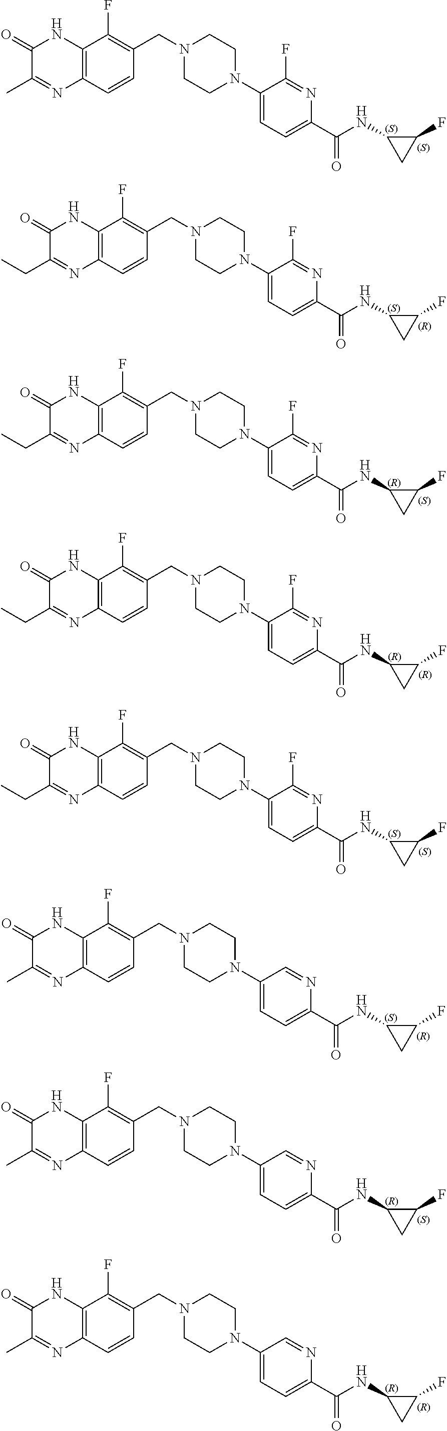

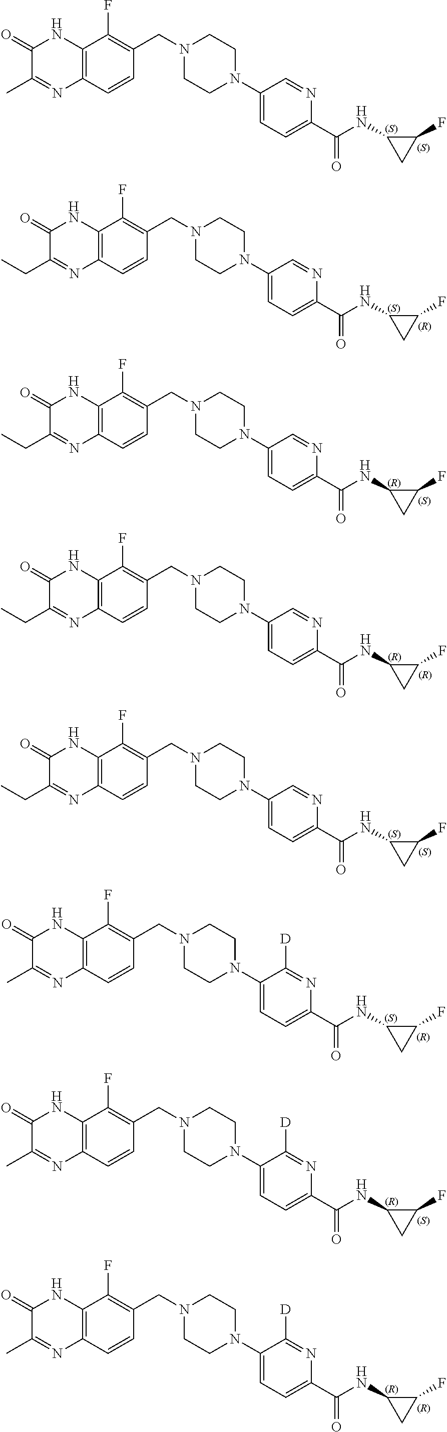

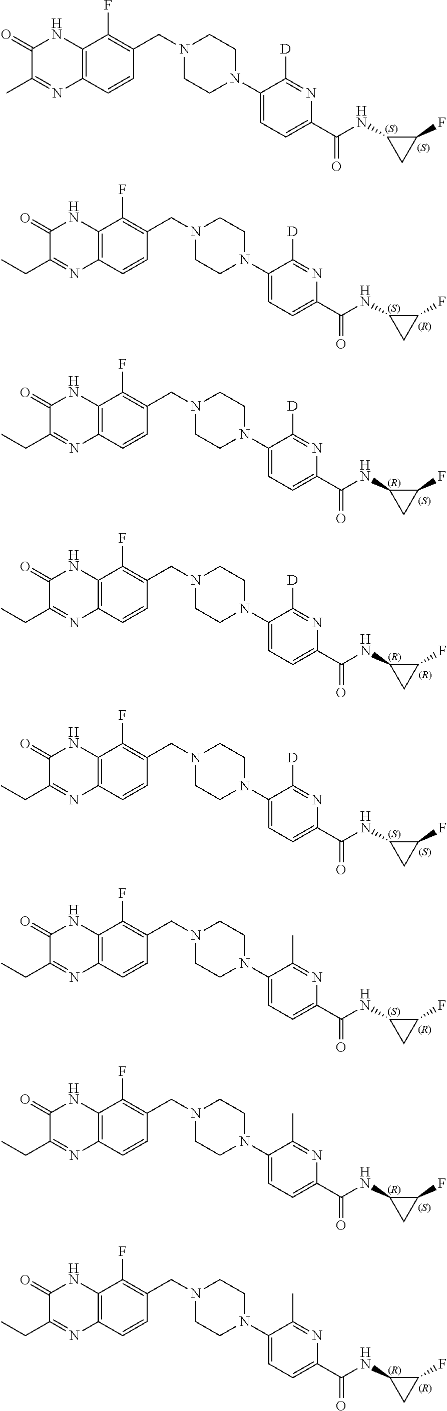

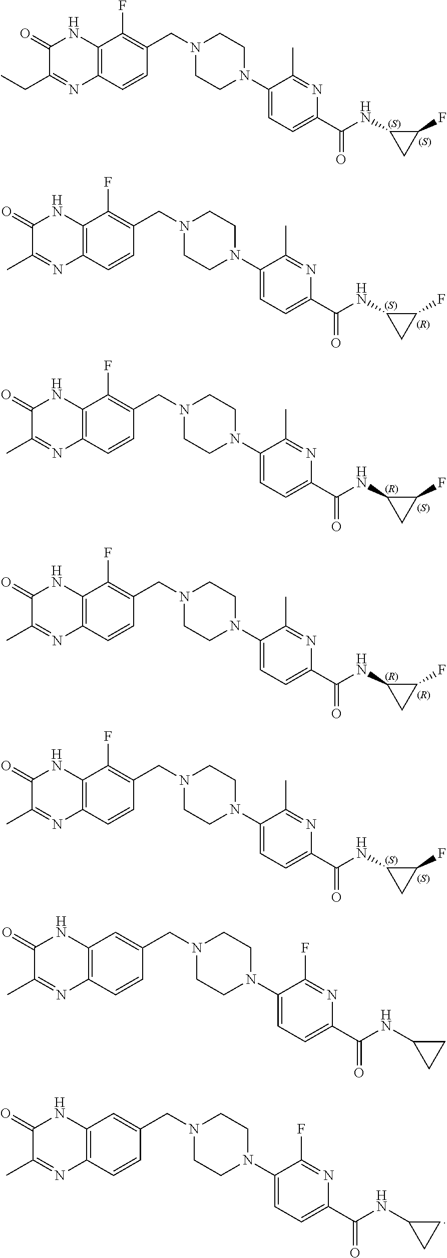

6. A pharmaceutical composition, comprising a pharmaceutically acceptable excipient or carrier, and the compound or the isomer thereof, the crystalline form thereof, the pharmaceutically acceptable salt thereof, the hydrate thereof, or the solvate thereof according to claim 1 as an active ingredient.

7. Use of the compound or the isomer thereof, the crystalline form thereof, the pharmaceutically acceptable salt thereof, the hydrate thereof, or the solvate thereof according to claim 1 in preparing a medicament for treating a related disease mediated by PARP.

8. The disease according to claim 7, wherein the disease is cancer, and the cancer is a hematologic cancer or a solid tumor, wherein the cancer is preferably ovarian cancer, breast cancer, endometrial cancer, cervical cancer, pancreatic cancer, prostate cancer, non-small cell lung cancer, small cell lung cancer, esophageal cancer, colorectal cancer, renal cell carcinoma, bladder cancer, melanoma, or leukemia.
